(19) United States
(12) Patent Application Publication
(10) Pub. No.: US 2025/0223623 A1
(43) Pub. Date: Jul. 10, 2025

(54) PRO-REGION MUTATIONS ENHANCING PROTEIN PRODUCTION IN GRAM-POSITIVE BACTERIAL CELLS

(71) Applicant: DANISCO US INC., Palo Alto, CA (US)

(72) Inventors: Cristina Bongiorni, Fremont, CA (US); Frits Goedegebuur, Vlaardingen (NL); Ryan L. Frisch, Newark, DE (US); Harm Mulder, Voorhout (NL)

(21) Appl. No.: 18/851,026

(22) PCT Filed: Mar. 30, 2023
(86) PCT No.: PCT/US2023/065162

§ 371 Date: Sep. 25, 2024

Related U.S. Application Data
(60) Provisional application No. 63/326,615, filed on Apr. 1, 2022.

(51) Int. Cl. C12P 21/02; C07K 14/32; C12N 1/20; C12N 15/11

(52) U.S. Cl. CPC C12P 21/02; C07K 14/32; C12N 1/20; C12N 15/11

(57) ABSTRACT

The instant disclosure is generally related to recombinant polynucleotides comprising novel pro-region DNA sequences. Certain aspects of the disclosure are related to recombinant Gram-positive bacterial strains comprising one or more introduced polynucleotides comprising novel pro-region DNA sequences operably linked to DNA sequences encoding proteins of interest.

### FIELD

The present disclosure is generally related to the fields of microbial host cells, molecular biology, protein engineering, fermentation, protein production, and the like. Certain aspects of the disclosure are related to novel pro-region nucleic acid (DNA) sequences, recombinant polynucleotides comprising novel pro-region DNA sequences, genetically modified (recombinant) Gram-positive bacterial strains comprising one or more introduced polynucleotides comprising novel pro-region DNA sequences operably linked to nucleic acid (DNA) sequences encoding proteins of interest and the like.

### CROSS REFERENCE TO REPLATED APPLICATIONS

This application claims benefit to U.S. Provisional Application No. 63/326,615, filed Apr. 1, 2022, which is hereby incorporated by reference in its entirety.

### REFERENCE TO A SEQUENCE LISTING

The contents of the electronic submission of the text file Sequence Listing, named “NB41862WOPCT_SequenceListing.xml” was created on Mar. 29, 2023, and is 51 KB in size, which is hereby incorporated by reference in its entirety.

### BACKGROUND

Gram-positive microorganisms are often used for large-scale industrial fermentation due to their ability to secrete their fermentation products into their culture media. Secreted proteins are exported across a cell membrane and a cell wall, and then subsequently released into the external media. For example, large-scale industrial fermentation and secretion of heterologous polypeptides is a widely used technique in industry, wherein microbial cells are transformed with a nucleic acid encoding a heterologous polypeptide to be expressed. Despite various advances in protein production methods, there remains a need in the art to provide more efficient methods for protein expression with the aim to enhance the production of proteins of interest which find use in the use in various industries.

### SUMMARY

As generally described herein, the instant disclosure provides, inter alia, compositions and methods for the production of proteins of interest in Gram-positive bacterial (host) cells. Certain embodiments are related to novel pro-region nucleic acid (DNA) sequences, recombinant polynucleotides (e.g., vectors, plasmids, expression cassettes, etc.) comprising novel pro-region DNA sequences, recombinant polynucleotides comprising novel pro-region sequences operably linked to downstream (3′) gene coding sequences, recombinant polynucleotides comprising novel pro-region sequences operably linked to upstream (5′) DNA sequences encoding pre-protein signal (secretion) sequences, and the like.

In certain aspects, the disclosure provides recombinant Gram-positive bacterial strains expressing one or more introduced polynucleotides encoding a protein of interest. In particular embodiments, one or more introduced polynucleotides comprise novel pro-region DNA sequences operably linked to DNA sequences encoding proteins of interest (which DNA sequences encoding proteins of interest may include upstream (5′) protein signal sequences operably linked thereto). In certain other aspects, the disclosure provide compositions and methods for the design/construction of recombinant Gram-positive bacterial strains expressing one or more introduced novel polynucleotide constructs encoding proteins of interest, compositions, and methods for cultivating recombinant strains expressing proteins of interest, compositions, and methods for the enhanced production of proteins of interest and the like.

For example, in one or more embodiments the disclosure provides novel variant pro-region sequences set forth in one or more amino acid sequences of SEQ ID NO: 9, SEQ ID NO: 11, SEQ ID NO: 14, SEQ ID NO: 29, SEQ ID NO: 30, SEQ ID NO: 32 and SEQ ID NO: 33, and/or combinations thereof. In other one or more embodiments, the disclosure provides novel variant pro-region sequences set forth in FIG. 1, FIG. 2, or FIG. 3, and/or combinations thereof. In yet other one or more embodiments, the disclosure provides novel variant pro-region sequences set forth or described in one of TABLES 1-5, and/or combinations thereof.

Certain one or more embodiments are therefore related to variant (mutant) pro-region sequences comprising one or more amino acid substitutions, one or more amino acid insertions, and the like. In certain embodiments, a variant pro-region sequence comprises an amino acid substitutions at position 30, wherein the amino acid positions of the variant pro-region are numbered according to SEQ ID NO: 15. In other embodiments, a variant pro-region sequence comprises an amino acid substitutions at position 30 and one or more positions selected from 1, 2, 3, 4, 6, 14, 16, 19, 20, 23, 36, 37, 38, 39, 42, 43, 44, 49, 50, 64, 65, 67, 68, 71, 79, 83 and 84, wherein the amino acid positions of the variant pro-region are numbered according to SEQ ID NO: 15. In one or more other embodiments, a variant pro-region sequence is derived from a parent or reference polypeptide comprising at least about 70%, 75%, 80%, 85%, 86%, 87%, 88%, 89%, 90%, 91%, 92%, 93%, 94%, 95%, 96%, 97%, 98%, 99%, or 100% sequence identity to positions 1-84 of SEQ ID NO: 15. In certain other embodiments, a variant pro-region sequence comprises amino acid insertions of at least glycine (G) at position 2 and lysine (K) at position 3, wherein the amino acid positions of the variant pro-region are numbered according to SEQ ID NO: 14. In other one or more embodiments, a variant pro-region sequence comprises amino acid insertions of at least glycine (G) at position 2 and lysine (K) at position 3, and amino acid substitutions at one or more positions selected from 1, 32, 38, 46, 66, 67, 70 and 73, wherein the amino acid positions of the variant pro-region are numbered according to SEQ ID NO: 14. In some other embodiments, a variant pro-region is derived from a parent or reference polypeptide comprising at least about 70%, 75%, 80%, 85%, 86%, 87%, 88%, 89%, 90%, 91%, 92%, 93%, 94%, 95%, 96%, 97%, 98%, 99%, or 100% amino acid sequence identity to positions 1-86 of SEQ ID NO: 14. In yet other embodiments, a variant pro-region sequence comprises amino acid insertions of at least glycine (G) at position 2, lysine (K) at position 3, and alanine (A) at position 4, wherein the amino acid positions of the variant pro-region are numbered according to SEQ ID NO: 30. In certain aspects, a variant pro-region is derived from a parent or reference polypeptide with at least 70%, 75%, 80%, 85%, 86%, 87%, 88%, 89%, 90%, 91%, 92%, 93%, 94%, 95%, 96%, 97%, 98%, 99%, or 100% amino acid sequence identity to positions 1-87 of SEQ ID NO: 30. In certain other one or more embodiments, a variant pro-region sequence comprises amino acid insertions of at least glycine (G) at position 2, lysine (K) at position 3, and serine (S) at position 4, wherein the amino acid positions of the variant pro-region are numbered according to SEQ ID NO: 29. In some other embodiments, a variant pro-region is derived from a parent or reference polypeptide with at least 70%, 75%, 80%, 85%, 86%, 87%, 88%, 89%, 90%, 91%, 92%, 93%, 94%, 95%, 96%, 97%, 98%, 99%, or 100% amino acid sequence identity to positions 1-87 of SEQ ID NO: 29. In other one or more embodiments, a variant pro-region sequence comprising amino acid insertions of at least glycine (G) at position 2, lysine (K) at position 3, alanine (A) at position 4, and alanine (A) at position 5, wherein the amino acid positions of the variant pro-region are numbered according to SEQ ID NO: 31. In certain one or more embodiments or aspects, a variant pro-region is derived from a parent or reference polypeptide with at least 70%, 75%, 80%, 85%, 86%, 87%, 88%, 89%, 90%, 91%, 92%, 93%, 94%, 95%, 96%, 97%, 98%, 99%, or 100% amino acid sequence identity to positions 1-88 of SEQ ID NO: 31. In other embodiments, a variant pro-region sequence comprises a glutamic acid (E) to glycine (G) substitution at position 30 (E30G), a leucine (L) to lysine (K) substitution at position 68 (L68K) and an isoleucine (I) to valine (V) substitution at position 73 (I72V), wherein the amino acid positions of the variant pro-region are numbered according to SEQ ID NO: 32. In other one or more aspects or embodiments, a variant pro-region sequence comprises a glutamic acid (E) to glycine (G) substitution at position 30 (E30G), a leucine (L) to lysine (K) substitution at position 68 (L68K) and glutamic acid (E) to isoleucine (I) substitution at position 80 (E80I), wherein the amino acid positions of the variant pro-region are numbered according to SEQ ID NO: 33. In certain embodiments, a variant pro-region is derived from a parent or reference polypeptide with at least 70%, 75%, 80%, 85%, 86%, 87%, 88%, 89%, 90%, 91%, 92%, 93%, 94%, 95%, 96%, 97%, 98%, 99%, or 100% amino acid sequence identity to positions 1-84 of SEQ ID NO: 15, SEQ ID NO: 32 and/or SEQ ID NO: 33. In other aspects, a variant pro-region of the disclosure comprises an amino acid modification set for in any one of TABLES 1-5, FIG. 1, FIG. 2, FIG. 3, SEQ ID NO: 9, SEQ ID NO: 11, SEQ ID NO: 14, SEQ ID NO: 29, SEQ ID NO: 30, SEQ ID NO: 32, SEQ ID NO: 33, and combinations thereof. In one or more embodiments, a variant pro-region comprising an amino acid modification set for in any one of TABLES 1-5, FIG. 1, FIG. 2, FIG. 3, SEQ ID NO: 9, SEQ ID NO: 11, SEQ ID NO: 14, SEQ ID NO: 29, SEQ ID NO: 30, SEQ ID NO: 32, SEQ ID NO: 33, and combinations thereof, is derived from a parent or reference polypeptide with at least 70%, 75%, 80%, 85%, 86%, 87%, 88%, 89%, 90%, 91%, 92%, 93%, 94%, 95%, 96%, 97%, 98%, 99%, or 100% amino acid sequence identity to SEQ ID NO: 15.

Thus, certain other one or more embodiments provide recombinant nucleic acids encoding novel variant pro-region sequences of the disclosure. Certain embodiments are therefore related to polynucleotides comprising variant pro-region nucleic acids and the like. In one or more embodiments, the disclosure provides polynucleotides comprising an upstream (5′) nucleic acid (sequence) encoding a variant pro-region of the disclosure operably linked to a downstream (3′) nucleic acid sequence encoding a heterologous protein of interest (POI). In certain other aspects, the disclosure provides polynucleotides comprising an upstream (5′) nucleic acid encoding a pre-protein signal (secretion) sequence operably linked to the downstream (3′) nucleic acid sequence encoding a variant pro-region of the disclosure operably linked to a downstream nucleic acid sequence encoding a protein of interest (POI). Certain other embodiments are related to expression cassettes comprising an upstream (5′) promoter region sequence operably linked to a downstream (3′) polynucleotide of the disclosure.

In certain other one or more embodiments, the disclosure is related to recombinant (modified) Gram-positive bacterial cells/strains comprising one or more introduced polynucleotides or expression cassettes of the disclosure. Thus, other embodiments of the disclosure are related to methods/processes for producing heterologous proteins of interest in recombinant Gram-positive cells set forth and described herein.

In some one or more embodiments, the disclosure provides methods for producing a heterologous POI in a Gram-positive bacterial cell comprising introducing into a Gram-positive cell an expression cassette comprising an upstream promoter region operably linked to a downstream nucleic acid sequence encoding a variant pro-region comprising amino acid substitutions at position 30 and one or more positions selected from 1, 2, 3, 4, 6, 14, 16, 19, 20, 23, 36, 37, 38, 39, 42, 43, 44, 49, 50, 64, 65, 67, 68, 71, 79, 83 and 84, wherein the variant pro-region nucleic acid (sequence) is operably linked to a downstream nucleic acid sequence encoding the POI, wherein the amino acid positions of the variant pro-region are numbered according to SEQ ID NO: 15, and growing/cultivating/fermenting the modified cell under conditions for the production of the POI. In certain other embodiments, the disclosure provides methods for producing a heterologous POI in a Gram-positive bacterial cell comprising introducing into a Gram-positive cell an expression cassette comprising an upstream promoter region sequence operably linked to a downstream nucleic acid (sequence) encoding a variant pro-region comprising amino acid insertions of glycine (G) at position 2 and lysine (K) at position 3, and amino acid substitutions at one or more positions selected from 1, 32, 38, 46, 66, 67, 70 and 73, wherein the variant pro-region nucleic acid (sequence) is operably linked to a downstream nucleic acid sequence encoding the POI, wherein the amino acid positions of the variant pro-region are numbered according to SEQ ID NO: 14, and growing/cultivating/fermenting the modified cell under conditions for the production of the POI. In yet other embodiments, the disclosure is related to methods for producing a heterologous POI in a Gram-positive bacterial cell comprising introducing into a Gram-positive cell an expression cassette of the disclosure, and growing/cultivating/fermenting the modified cell under conditions for the production of the POI. In certain one or more embodiments of the methods, the cassette comprises a nucleic acid (sequence) encoding a pre-protein signal (secretion) sequence operably linked and positioned between the promoter and variant pro-region sequences. In some other embodiments of the methods, the modified cell produces an increased amount of the POI relative to a control Gram-positive cell. For example, a control Gram-positive cell comprising an introduced expression cassette comprising the same upstream promoter sequence (i.e., same as the modified cell) operably linked to a downstream nucleic acid encoding a pro-region sequence comprising SEQ ID NO: 15 operably linked to a downstream nucleic acid sequence encoding the same POI (i.e., same POI as the modified cell). In certain embodiments of the methods, the introduced cassette is integrated into the genome of the cell. In certain embodiments of the methods, at least two cassettes are introduced into the cell. Certain other one or more embodiments of the disclosure are described herein and set forth below.

### BRIEF DESCRIPTION OF DRAWINGS

FIG. 1 presents the amino acid sequence of a wild-type (reference) B. lentus pro-region and certain SEL variant pro-region sequences of the disclosure. As shown in FIG. 1, the reference B. lentus pro-region sequence comprises eighty-four (84) amino acid (residue) positions set forth in SEQ ID NO: 15 (wherein the glutamic acid (E) residue at position 30 is underlined), variant pro-region sequence A comprises eighty-four (84) amino acid positions set forth in SEQ ID NO: 9 (wherein the histidine (H) residue at position 30 is bold), variant pro-region sequence B comprises eighty-four (84) amino acid positions set forth in SEQ ID NO: 11 (wherein the glycine (G) residue at position 30 is bold), variant pro-region sequence C comprises eighty-six (86) amino acid positions set forth in SEQ ID NO: 14 (wherein the glycine (G) and lysine (K) residues inserted at positions 2 and 3 respectively, are shown in bold (GK), and the E residue at position 32 is underlined), variant pro-region sequence D comprises eighty-seven (87) amino acid positions set forth in SEQ ID NO: 29 (wherein the glycine (G), lysine (K) and serine (S) residues inserted at positions 2-4 respectively, are shown in bold (GKS)), variant pro-region sequence E comprises eighty-seven (87) amino acid positions set forth in SEQ ID NO: 30 (wherein the glycine (G), lysine (K) and alanine (A) residues inserted at positions 2-4 respectively, are shown in bold (GKA)), variant pro-region sequence F comprises eighty-eight (88) amino acid positions set forth in SEQ ID NO: 31 (wherein the glycine (G), lysine (K), alanine (A) and alanine (A) residues inserted at positions 2-5 respectively, are shown in bold (GKAA)), variant pro-region sequence G comprises eighty-four (84) amino acid positions set forth in SEQ ID NO: 32 (wherein the glycine (G) residue at position 30 is bold, with the L68K and 172V substitutions underlined) and variant pro-region sequence H comprises eighty-four (84) amino acid positions set forth in SEQ ID NO: 33 (wherein the glycine (G) residue at position 30 is bold, with the L68K and E80I substitutions underlined).

FIG. 2 shows the amino acid sequences of variant pro-region sequence A (SEQ 09), variant pro-region sequence B (SEQ 11), variant pro-region sequence C (SEQ 14), variant pro-region sequence D (SEQ 29), variant pro-region sequence E (SEQ 30), variant pro-region sequence F (SEQ 31), variant pro-region sequence G (SEQ 32) and variant pro-region sequence H (SEQ 33) aligned with the wild-type (WT) pro-region sequence (SEQ 15). As presented in FIG. 2, the WT pro-region comprises a glutamic acid (E) residue at position 30 (E; SEQ 15), variant pro-region sequence A comprises a histidine (H) residue at position 30 (H; SEQ 09), variant pro-region sequence B comprises a glycine (G) residue at position 30 (G; SEQ 11), variant pro-region sequence C comprises the two (2) amino acid insertion of glycine (G) and lysine (K)(GK insertion; SEQ 14), variant pro-region sequence D comprises the three (3) amino acid insertion of glycine (G), lysine (K) and serine (S) (GKS insertion; SEQ 29), variant pro-region sequence E comprises the three (3) amino acid insertion of glycine (G), lysine (K) and alanine (A) (GKA insertion; SEQ 30), variant pro-region sequence F comprises the four (4) amino acid insertion of glycine (G), lysine (K), alanine (A), alanine (A) (GKAA insertion; SEQ 31), variant pro-region sequence G comprises a glycine (G) residue at position 30, a lysine (K) residue at position 68 and a valine (V) residue at position 72, and variant pro-region sequence H comprises a glycine (G) residue at position 30, a lysine (K) residue at position 68 and an isoleucine (I) residue at position 80. For example, as shown in the alignment of the WT pro-region sequence (SEQ 15) and variant pro-region sequence C (SEQ 14), the two (2) amino acid residues GK have been inserted between the alanine (A) at position 1 and glutamic acid (E) at position 2 of the WT pro-region (SEQ ID NO: 15), resulting in the 86 amino acid variant pro-region sequence C of SEQ ID NO: 14. As presented in FIG. 2, the two-hyphens (--) shown in SEQ 15, SEQ 09, SEQ 11, SEQ 32 and SEQ 33, indicate a two (2) amino acid position gap relative to variant pro-region sequence C shown in SEQ 14.

FIG. 3 shows the amino acid (residue) position numbering of the reference (wild-type) B. lentus pro-region amino acid sequence (E₃₀, SEQ ID NO: 15), variant pro-region sequence A (H₃₀; SEQ ID NO: 09), variant pro-region sequence B (G₃₀; SEQ ID NO: 11), variant pro-region sequence C (G₂K₃ insertion, SEQ ID NO: 14), variant pro-region sequence D (G₂K₃S₄ insertion, SEQ ID NO: 29), variant pro-region sequence E (G₂K₃A₄ insertion, SEQ ID NO: 30), variant pro-region sequence F (G₂K₃A₄A₅ insertion, SEQ ID NO: 31), variant pro-region sequence G (with E30G, L68K and I72V substitutions; SEQ ID NO: 32) and variant pro-region sequence F (with E30G, L68K and E80I substitutions; SEQ ID NO: 33), wherein the amino acid positions are indicated with subscript numerals in the 5′ (N-term) to 3′ (C-term) direction, e.g., alanine (A₁) at position 1 of SEQ ID NO: 15, glutamic acid (E₂) at position 2 of SEQ ID NO: 15, etc. For example, mutant pro-region sequences of the disclosure may refer to the wild-type (reference) pro-region sequence numbering (SEQ ID NO: 15, E30), the variant (reference) pro-region sequence A numbering (SEQ ID NO: 9, H30), the variant (reference) pro-region sequence B numbering (SEQ ID NO: 11, G30), the variant (reference) pro-region sequence C numbering (SEQ ID NO: 14, GK insertion), the variant (reference) pro-region sequence D numbering (SEQ ID NO: 29, GKS insertion), the variant (reference) pro-region sequence E numbering (SEQ ID NO: 20, GKA insertion), the variant (reference) pro-region sequence F numbering (SEQ ID NO: 31, GKAA insertion), the variant (reference) pro-region sequence G numbering (SEQ ID NO: 32, L68K/I72V substitutions), the variant (reference) pro-region sequence H numbering (SEQ ID NO: 33, L68K/E80I substitutions), or a combination thereof, as set forth in FIG. 3.

### BRIEF DESCRIPTION OF THE BIOLOGICAL SEQUENCES

SEQ ID NO: 1 is a nucleic acid (DNA) sequence comprising an upstream (5′) aprE gene flanking region, a variant of B. subtilis rrn1-P2 promoter and 5′-aprE UTR region.

SEQ ID NO: 2 is the amino acid sequence of a wild-type Bacillus gibsonii subtilisin named “BG46”.

SEQ ID NO: 3 is the amino acid sequence of a variant B. gibsonii BG46 subtilisin named “BG46_variant 1”.

SEQ ID NO: 4 is a DNA sequence encoding a wild-type B. subtilis AprE protein signal sequence.

SEQ ID NO: 5 is a DNA sequence encoding variant pro-region sequence A (30H, SEQ ID NO: 9).

SEQ ID NO: 6 is the DNA sequence of a wild-type B. amyloliquefaciens BPN′ terminator.

SEQ ID NO: 7 is the DNA sequence of a kanamycin (kan) gene expression cassette.

SEQ ID NO: 8 is the amino acid sequence of a variant B. gibsonii BG46 subtilisin named “BG46_variant 2”.

SEQ ID NO: 9 is the amino acid sequence encoded by the variant pro-region A DNA sequence (SEQ ID NO: 5).

SEQ ID NO: 10 is a DNA sequence encoding variant pro-region sequence B (30G, SEQ ID NO: 11).

SEQ ID NO: 11 is the amino acid sequence encoded by the variant pro-region B DNA sequence (SEQ ID NO: 10).

SEQ ID NO: 12 is the amino acid sequence of a B. amyloliquefaciens BPN′ pro-region sequence

SEQ ID NO: 13 is a DNA sequence encoding a variant pro-region sequence C (GK insertion, SEQ ID NO: 14).

SEQ ID NO: 14 is the amino acid sequence encoded by the variant pro-region C DNA sequence (SEQ ID NO: 13).

SEQ ID NO: 15 is the amino acid sequence of the wild-type (WT) B. lentus pro-region sequence.

SEQ ID NO: 16 is a DNA sequence of the B. licheniformis serA gene 5′ FR.

SEQ ID NO: 17 is a DNA sequence comprising a rrn1-p3 promoter region.

SEQ ID NO: 18 is the DNA sequence of a B. subtilis aprE 5′-UTR.

SEQ ID NO: 19 is the DNA sequence of a B. licheniformis amyL terminator.

SEQ ID NO: 20 is a DNA sequence of the B. licheniformis serA gene 3′ FR

SEQ ID NO: 21 is a DNA sequence of the B. licheniformis lysA gene 5′ FR.

SEQ ID NO: 22 is a DNA sequence of the B. licheniformis lysA gene 3′ FR.

SEQ ID NO: 23 is a DNA sequence encoding a variant pro-region sequence C (GKA insertion, SEQ ID NO: 14).

SEQ ID NO: 24 is the amino acid sequence encoded by the variant pro-region C DNA sequence (SEQ ID NO: 23).

SEQ ID NO: 25 is a DNA sequence encoding a variant pro-region sequence C (GKAA insertion, SEQ ID NO: 14).

SEQ ID NO: 26 is the amino acid sequence encoded by the variant pro-region C DNA sequence (SEQ ID NO: 25).

SEQ ID NO: 27 is a DNA sequence encoding a variant pro-region sequence C (GKAS insertion, SEQ ID NO: 14).

SEQ ID NO: 28 is the amino acid sequence encoded by the variant pro-region C DNA sequence (SEQ ID NO: 27).

SEQ ID NO: 29 is the amino acid sequence of variant pro-region sequence D.

SEQ ID NO: 30 is the amino acid sequence of variant pro-region sequence E.

SEQ ID NO: 31 is the amino acid sequence of variant pro-region sequence F.

SEQ ID NO: 32 is the amino acid sequence of variant pro-region sequence G.

SEQ ID NO: 33 is the amino acid sequence of variant pro-region sequence H.

### DETAILED DESCRIPTION

As briefly set forth above and described hereinafter in detail, the present disclosure provides, inter alia, novel pro-region nucleic acid (DNA) sequences, recombinant polynucleotides (e.g., vectors, plasmids, expression cassettes, etc.) comprising novel pro-region DNA sequences, recombinant polynucleotides comprising novel pro-region sequences operably linked to downstream (3′) gene coding sequences, recombinant polynucleotides comprising novel pro-region sequences operably linked to upstream (5′) DNA sequences encoding protein signal (secretion) sequences, and the like. In certain aspects, the disclosure provides recombinant Gram-positive bacterial strains expressing one or more introduced polynucleotides encoding a protein of interest. In particular embodiments, one or more introduced polynucleotides comprise novel pro-region DNA sequences operably linked to DNA sequences encoding proteins of interest (which DNA sequences encoding proteins of interest may include upstream (5′) protein signal sequences operably linked thereto). Thus, certain other aspects of the disclosure provide, inter alia, compositions and methods for the design/construction of recombinant Gram-positive bacterial strains expressing one or more introduced novel polynucleotide constructs encoding proteins of interest, compositions and methods for cultivating recombinant strains expressing proteins of interest, compositions and methods for the enhanced production of proteins of interest and the like.

### I. DEFINITIONS

In view of the recombinant (modified) strains of the disclosure and methods thereof described herein, the following terms and phrases are defined. Terms not defined herein should be accorded their ordinary meaning as used in the art.

Unless defined otherwise, all technical and scientific terms used herein have the same meaning as commonly understood by one of ordinary skill in the art to which the present compositions and methods apply. Although any methods and materials similar or equivalent to those described herein can also be used in the practice or testing of the present compositions and methods, representative illustrative methods and materials are now described. All publications and patents cited herein are incorporated by reference in their entirety.

It is further noted that the claims may be drafted to exclude any optional element. As such, this statement is intended to serve as antecedent basis for use of such exclusive terminology as “solely,” “only”, “excluding”, “not including” and the like, in connection with the recitation of claim elements, or use of a “negative” limitation or proviso thereof.

As will be apparent to those of skill in the art upon reading this disclosure, each of the individual embodiments described and illustrated herein has discrete components and features which may be readily separated from or combined with the features of any of the other several embodiments without departing from the scope or spirit of the present compositions and methods described herein. Any recited method can be carried out in the order of events recited or in any other order which is logically possible.

As used herein, the phrases “Gram-positive bacteria”, Gram-positive cells” “Gram-positive bacterial strains”, and/or “Gram positive bacterial cells” have the same meaning as used in the art. For example, Gram-positive bacterial cells include all strains of Actinobacteria and Firmicutes. In certain embodiments, such Gram-positive bacteria are of the classes Bacilli, Clostridia and Mollicutes.

As used herein, the genus “Bacillus” includes all species within the genus “Bacillus′” as known to those of skill in the art, including but not limited to B. subtilis, B. licheniformis, B. lentus, B. brevis, B. stearothermophilus, B. alkalophilus, B. amyloliquefaciens, B. clausii, B. halodurans, B. megaterium, B. coagulans, B. circulans, B. lautus, and B. thuringiensis. It is recognized that the genus Bacillus continues to undergo taxonomical reorganization. Thus, it is intended that the genus include species that have been reclassified, including but not limited to such organisms as B. stearothermophilus, which is now named “Geobacillus stearothermophilus”.

As used herein, the terms “recombinant” or “non-natural” refer to an organism, microorganism, cell, nucleic acid molecule, or vector that has at least one engineered genetic alteration, or has been modified by the introduction of a heterologous nucleic acid molecule, or refer to a cell (e.g., a microbial cell) that has been altered such that the expression of a heterologous or endogenous nucleic acid molecule or gene can be controlled. Recombinant also refers to a cell that is derived from a non-natural cell or is progeny of a non-natural cell having one or more such modifications. Genetic alterations include, for example, modifications introducing expressible nucleic acid molecules encoding proteins, or other nucleic acid molecule additions, deletions, substitutions, or other functional alteration of a cell's genetic material. For example, recombinant cells may express genes or other nucleic acid molecules that are not found in identical or homologous form within a native (wild-type) cell (e.g., a fusion or chimeric protein), or may provide an altered expression pattern of endogenous genes, such as being over-expressed, under-expressed, minimally expressed, or not expressed at all. “Recombination”, “recombining” or generating a “recombined” nucleic acid is generally the assembly of two or more nucleic acid fragments wherein the assembly gives rise to a chimeric gene.

The term “derived” encompasses the terms “originated” “obtained,” “obtainable,” and “created,” and generally indicates that one specified material or composition finds its origin in another specified material or composition, or has features that can be described with reference to the another specified material or composition. For example, recombinant Gram-positive bacterial cells of the disclosure may be derived/obtained from any known Gram-positive bacterial strains.

As used herein, “nucleic acid” refers to a nucleotide or polynucleotide sequence, and fragments or portions thereof, as well as to DNA, cDNA, and RNA of genomic or synthetic origin, which may be double-stranded or single-stranded, whether representing the sense or antisense strand. It will be understood that as a result of the degeneracy of the genetic code, a multitude of nucleotide sequences may encode a given protein.

It is understood that the polynucleotides (or nucleic acid molecules) described herein include “genes”, “vectors” and “plasmids”.

Accordingly, the term “gene”, refers to a polynucleotide that codes for a particular sequence of amino acids, which comprise all, or part of a protein coding sequence, and may include regulatory (non-transcribed) DNA sequences, such as promoter sequences, which determine for example the conditions under which the gene is expressed. The transcribed region of the gene may include untranslated regions (UTRs), including 5′-untranslated regions (UTRs), and 3′-UTRs, as well as the coding sequence.

As used herein, an “endogenous gene” refers to a gene in its natural location in the genome of an organism.

As used herein, a “heterologous” gene, a “non-endogenous” gene, or a “foreign” gene refer to a gene not normally found in the host organism, but that is introduced into the host organism by gene transfer. The term “foreign” gene(s) comprises native genes inserted into a non-native organism and/or chimeric genes inserted into a native or non-native organism.

As used herein, a “heterologous control sequence”, refers to a gene expression control sequence (e.g., promoters, enhancers, terminators, etc.) which does not function in nature to regulate (control) the expression of the gene of interest. Generally, heterologous nucleic acids are not endogenous (native) to the cell, or a part of the genome in which they are present, and have been added to the cell, by infection, transfection, transduction, transformation, microinjection, electroporation, and the like. A “heterologous” nucleic acid construct may contain a control sequence/DNA coding (ORF) sequence combination that is the same as, or different, from a control sequence/DNA coding sequence combination found in the native host cell.

As used herein, the term “expression” refers to the transcription and stable accumulation of sense (mRNA) or anti-sense RNA, derived from a nucleic acid molecule of the disclosure. Expression may also refer to translation of mRNA into a polypeptide. Thus, the term “expression” includes any steps involved in the production of the polypeptide including, but not limited to, transcription, post-transcriptional modification, translation, post-translational modification, secretion and the like.

As used herein, the term “coding sequence” refers to a nucleotide sequence, which directly specifies the amino acid sequence of its (encoded) protein product. The boundaries of the coding sequence are generally determined by an open reading frame (hereinafter, “ORF”), which usually begins with an ATG start codon. The coding sequence typically includes DNA, cDNA, and recombinant nucleotide sequences.

As used herein, the terms “promoter”, “promoter element”, “promoter sequence” and the like, refer to a nucleic acid (DNA) sequence capable of controlling the transcription of a gene coding sequence (CDS) into messenger RNA (mRNA) when the promoter region sequence is placed upstream (5′) and operably linked to the downstream (3′) gene CDS. As generally understood by of skilled in the art, promoters typically provide a site for specific binding by RNA polymerase and the initiation of transcription. In certain aspects, the term “promoter” refers to the minimal portion of the promoter nucleic acid sequence required to initiate transcription (i.e., comprising RNA polymerase binding sites). For example, a promoter generally comprises a “−10” (consensus sequence) element and a “−35” (consensus sequence) element, which are upstream (5′) and relative to the +1 transcription start site (TSS) of the gene CDS to be transcribed. The core promoter −10 and −35 elements are generally referred to in the art as the “TATAAT” (Pribnow box) consensus region and the “TTGACA” consensus region, respectively. The spacing of the core promoter (10 and −35) regions are generally separated (spaced) by about fifteen-twenty (15-20) intervening base pairs (nucleotides).

Promoters may be derived in their entirety from a native gene, or be composed of different elements derived from different promoters found in nature, or even comprise synthetic nucleic acid segments. It is understood by those skilled in the art that different promoters may direct the expression of a gene in different cell types, or at different stages of development, or in response to different environmental or physiological conditions. Promoters can be constitutive promoters, inducible promoters, tunable promoters, hybrid promoters, synthetic promoters, tandem promoters, etc. Promoters which cause a gene to be expressed in most cell types at most times are commonly referred to as “constitutive promoters”. It is further recognized that since in most cases the exact boundaries of regulatory sequences have not been completely defined, DNA fragments of different lengths may have identical promoter activity. In certain aspects, an upstream (5′) promoter sequence (pro) operably linked to a downstream DNA sequence encoding a protein signal sequence (SS) operably linked to a downstream DNA sequence encoding a pro-region sequence (PRO) operably linked to a downstream (3′) DNA sequence (ORF) encoding a mature protein of interest, may be schematically presented as, 5′-[pro]-[SS]-[PRO]-[ORF]-3′.

As used herein, a “functional promoter sequence controlling the expression of a gene of interest linked to the gene of interest's protein coding sequence” refers to a promoter sequence which controls the transcription and translation of the coding sequence in a desired Gram-positive host cell. For example, in certain embodiments, the present disclosure provides polynucleotides comprising an upstream (5′) promoter (or 5′ promoter region, or tandem 5′ promoters and the like) functional in a Gram-positive cell, wherein the functional promoter region is operably linked to a nucleic acid sequence encoding a protein of interest.

As used herein, the term “precursor protein” refers to an inactive form of a protein. In certain aspects, a full-length protein is synthesized as precursor, in the form of a pro-sequence and mature protein (abbreviated, “pre-protein”). In other aspects, a full-length protein is synthesized as precursor, in the form of a signal peptide sequence, a pro-sequence and mature protein (abbreviated, “pre-pro-protein”). For example, pre-sequences usually act as signal peptides for transport, and pro-sequences are typically essential for the correct folding of the associated (mature) protein.

As used herein, the term “mature protein” refers to an active form of a protein, in contrast to the inactive precursor (full-length) protein.

As used herein, the terms “signal sequence”, “secretion signal” and “signal peptide” may be used interchangeably and refer to a sequence of amino acid residues that may participate in the secretion or direct transport of a precursor protein. The signal (pre) sequence is typically cleaved from the precursor protein by a signal peptidase during translocation. The signal (pre) sequence is typically located N-terminal to the mature protein sequence, or located N-terminal to the pro-region (pro) sequence when a signal (pre) sequence and a pro-region (pro) sequence are used in operable combination and upstream (5′) of the mature POI sequence.

As used herein, the terms “pro sequence”, “pro-sequence” and “pro-region sequence” may be used interchangeably and abbreviated as “PRO” sequence, “Pro” sequence, “pro” sequence and the like. The term pro-sequence as used herein has the same meaning as understood in the art. For example, the B. subtilis alkaline serine protease “subtilisin” is first produced as a pre-pro-subtilisin, which consists of a signal (pre) sequence for protein secretion followed by a seventy-seven (77) amino acid pro-region (pro) sequence followed by the amino acid sequence encoding the mature subtilisin (e.g., pre-pro-subtilisin).

Pro-sequences are often essential for the correct folding of the associated (mature) protein, acting as an intra-molecular chaperone (e.g., catalyzing the protein-folding reaction directly). Likewise, pro-sequences may be required for both folding and intracellular transport (or secretion) of the mature protein of interest, suggesting that these two functionalities are intimately related. In certain aspects, a pro-region sequence of the disclosure comprises an amino acid sequence derived from a wild-type (WT, reference) B. lentus pro-region sequence of SEQ ID NO: 15. In certain embodiments, a pro-region sequence is derived from an amino acid sequence comprising homology to SEQ ID NO: 15, SEQ ID NO: 9, SEQ ID NO: 11, SEQ ID NO: 14, SEQ ID NO: 29, SEQ ID NO: 30, SEQ ID NO: 31, SEQ ID NO: 32 and/or SEQ ID NO: 33. Thus, in certain aspects, amino acid modifications of the one or more pro-region variants described herein are numbered by reference to a pro-region amino acid sequence of SEQ ID NO: 15, SEQ ID NO: 9, SEQ ID NO: 11 or SEQ ID NO: 14.

For example, the amino acid sequence of one or more pro-region variants described herein can be aligned with the amino acid sequence of SEQ ID NO: 15 using an alignment algorithm, and each amino acid residue in the given amino acid sequence that aligns (preferably optimally aligns) with an amino acid residue in SEQ ID NO: 15 is conveniently numbered by reference to the numerical position of that corresponding amino acid residue. Sequence alignment algorithms will identify the location or locations where insertions or deletions occur in a subject sequence when compared to a query sequence (also sometimes referred to as a “reference sequence”). Sequence alignment with other pro-region amino acid sequences can be determined using an amino acid alignment algorithms.

In certain aspects, reference to a pro-region amino acid sequence “position” may be presented as a single letter amino acid (residue) followed by the position number (e.g., SEQ ID NO: 15, alanine at position 1 presented as “A1”, glutamic acid at position 2 presented as “E2”, etc.). In related embodiments, reference to a variant (mutant) pro-region amino acid sequence may be presented as a single letter amino acid (residue), wherein the amino acid position of the parent (reference) sequence is numbered followed by the mutated amino acid (residue) at the same position (e.g., SEQ ID NO: 15, alanine (A) at position 1 substituted with a glycine (G) presented as “A1G”, glutamic acid (E) at position 30 substituted with a histidine (H) presented as “E30H”, etc.). Multiple amino acid residues may also be substituted at the same position of a pro-region amino acid sequence, wherein the amino acid position of the parent (reference) sequence is numbered followed by the mutated amino acid residue(s) at the same position separated by a fore slash (e.g., SEQ ID NO: 15, alanine (A) at position 1 substituted with glycine (G), glutamic acid (E) or lysine (K) may be presented as “A1G/E/K”, etc.).

Certain ranges are presented herein with numerical values being preceded by the term “about”. The term “about” is used herein to provide literal support for the exact number that it precedes, as well as a number that is near to or approximately the number that the term precedes. In determining whether a number is near to or approximately a specifically recited number, the near or approximating unrecited number can be a number which, in the context in which it is presented, provides the substantial equivalent of the specifically recited number. For example, in connection with a numerical value, the term “about” refers to a range of −10% to +10% of the numerical value, unless the term is otherwise specifically defined in context.

As used herein, a polynucleotide encoding a “PRO” sequence, a polynucleotide encoding a “pro-region” sequence and a polynucleotide encoding a “pro-sequence” may be used interchangeably, and refer to a DNA sequence positioned immediately upstream (5′) and operably linked to a downstream (3′) gene coding sequence (CDS) encoding a mature protein of interest (POI). In certain aspects, a polynucleotide encoding a pro-region sequence refers to a DNA sequence positioned and operably linked between an upstream (5′) protein signal sequence (SS) and a downstream (3′) gene CDS encoding a mature POI. For example, a pro-region DNA sequence (PRO) operably linked an upstream (5′) DNA sequence (SS) encoding protein signal sequence and operably linked to a downstream (3′) DNA gene CDS encoding the mature POI (e.g., 5′-[SS]-[PRO]-[ORF]-3′; N-[pre]-[pro]-[protein]-C).

As used herein, a “wild-type (WT) B. lentus pro-region” sequence comprises the amino acid sequence set forth in SEQ ID NO: 15. In certain aspects, the WT (reference) pro-region sequence (SEQ ID NO: 15) may be referred to as the “E30 pro-sequence” or the “E₃₀ pro-sequence”.

As used herein, a mutant pro-region sequence named “variant pro-region A” comprises the amino acid sequence set forth in SEQ ID NO: 9. In certain embodiments, the variant pro-region A sequence (SEQ ID NO: 9) may be referred to as the “H30 pro-sequence” or the “H₃₀ pro-sequence”.

As used herein, a mutant pro-region sequence named “variant pro-region B” comprises the amino acid sequence set forth in SEQ ID NO: 11. In certain embodiments, the variant pro-region B sequence (SEQ ID NO: 11) may be referred to as the “G30 pro-sequence” or the “G₃₀ pro-sequence”.

As used herein, a mutant pro-region sequence named “variant pro-region C” comprises the amino acid sequence set forth in SEQ ID NO: 14. In certain embodiments, the variant pro-region C sequence (SEQ ID NO: 14) may be referred to as the “GK insertion pro-sequence” or the “G₂K₃ insertion pro-sequence”.

As used herein, a mutant pro-region sequence named “variant pro-region D” comprises the amino acid sequence set forth in SEQ ID NO: 29. In certain embodiments, the variant pro-region D sequence (SEQ ID NO: 29) may be referred to as the “GKS insertion pro-sequence” or the “G₂K₃S₄ insertion pro-sequence”.

As used herein, a mutant pro-region sequence named “variant pro-region E” comprises the amino acid sequence set forth in SEQ ID NO: 30. In certain embodiments, the variant pro-region E sequence (SEQ ID NO: 30) may be referred to as the “GKA insertion pro-sequence” or the “G₂K₃A₄ insertion pro-sequence”.

As used herein, a mutant pro-region sequence named “variant pro-region F” comprises the amino acid sequence set forth in SEQ ID NO: 31. In certain embodiments, the variant pro-region F sequence (SEQ ID NO: 31) may be referred to as the “GKAA insertion pro-sequence” or the “G₂K₃A₄A₅ insertion pro-sequence”.

As used herein, a mutant pro-region sequence named “variant pro-region G” comprises the amino acid sequence set forth in SEQ ID NO: 32. In certain embodiments, the variant pro-region G sequence (SEQ ID NO: 32) may be referred to as the L68K/I72V pro-sequence.

As used herein, a mutant pro-region sequence named “variant pro-region H” comprises the amino acid sequence set forth in SEQ ID NO: 32. In certain embodiments, the variant pro-region H sequence (SEQ ID NO: 33) may be referred to as the L68K/E80I pro-sequence.

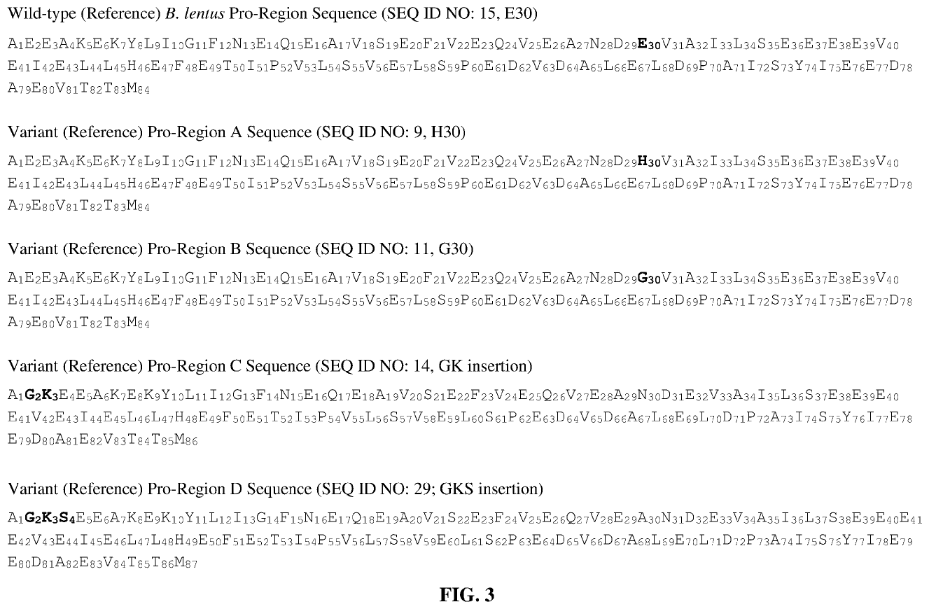

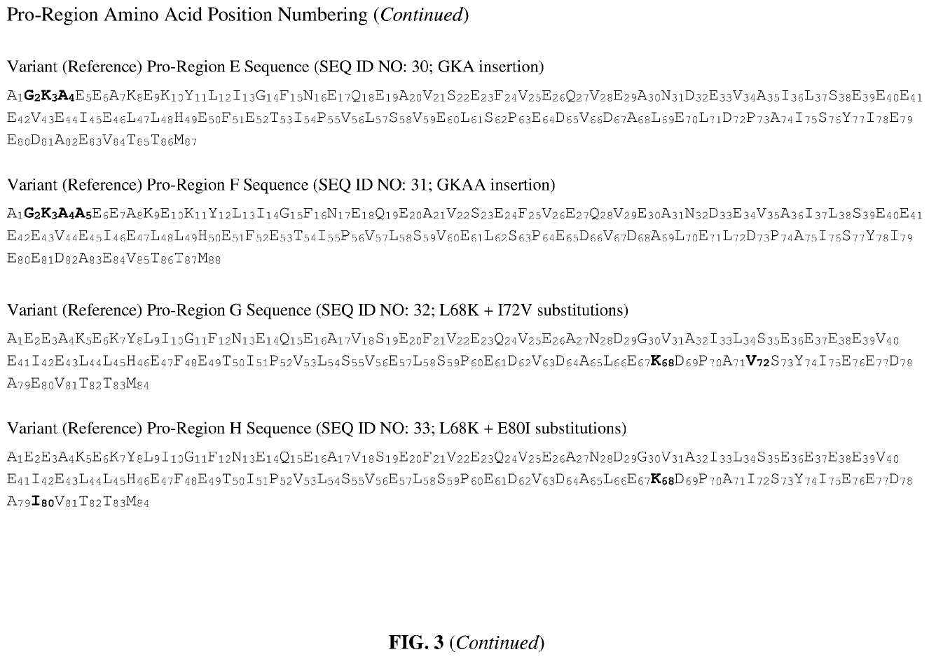

As generally shown in FIG. 3, the amino acid (residue) position numbering may be referenced to the WT pro-region amino acid sequence (E₃₀, SEQ ID NO: 15), the variant pro-region sequence A (H₃₀; SEQ ID NO: 09), the variant pro-region sequence B (G₃₀; SEQ ID NO: 11), the variant pro-region sequence C (GK insertion, SEQ ID NO: 14), the variant pro-region sequence D (GKS insertion, SEQ ID NO: 29), the variant pro-region sequence E (GKA insertion, SEQ ID NO: 30), the variant pro-region sequence F (GKAA insertion, SEQ ID NO: 31), the variant pro-region sequence G (L68K/I72V, SEQ ID NO: 32), and/or the variant pro-region sequence H (L68K/E80I, SEQ ID NO: 33).

As used herein, the phrase “polynucleotide encoding a full-length protein” refers to a DNA sequence encoding a “precursor” protein; and the phrase “polynucleotide encoding a mature protein” refers to a DNA sequence encoding a “mature” protein, as defined herein. In certain aspects, a polynucleotide encoding a precursor protein comprises at least an upstream (5′) DNA sequence encoding pro-region amino acid sequence operably linked to a downstream (3′) DNA sequence (e.g., open reading frame, ORF) encoding the amino acid sequence of a mature protein of interest (POI). In other aspects, a polynucleotide encoding a precursor protein comprises at least an upstream (5′) DNA sequence encoding a protein signal sequence operably linked to a downstream (3′) DNA sequence encoding a pro-region amino acid sequence operably linked to a downstream (3′) ORF encoding the amino acid sequence of a mature POI.

As used herein, the term “untranslated region” may be abbreviated “UTR”.

As used herein, the phrases “five prime (5′) untranslated region”, “5′ untranslated region” and/or “5′ transcript leader” may be used interchangeably and abbreviated as “5′-UTR”. As generally understood in the art, the 5′-UTR is known to be the region of a messenger RNA (mRNA) that is directly upstream (5′) from the initiation codon.

A nucleic acid is “operably linked” when it is placed into a functional relationship with another nucleic acid sequence. For example, DNA encoding a secretory leader (i.e., a signal sequence) is operably linked to DNA encoding a polypeptide if it is expressed as a pre-protein that participates in the secretion of the polypeptide; a promoter or enhancer is operably linked to a coding sequence (CDS, ORF) if it affects the transcription of the sequence; or a ribosome binding site is operably linked to a coding sequence if it is positioned so as to facilitate translation. Generally, “operably linked” means that the DNA sequences being linked are contiguous, and, in the case of a secretory leader, contiguous and in reading phase. However, enhancers do not have to be contiguous. Linking is accomplished by ligation at convenient restriction sites. If such sites do not exist, the synthetic oligonucleotide adaptors or linkers are used in accordance with conventional practice. Thus, the term operably linked generally refers to the association (juxtaposition) of nucleic acid sequences on a single nucleic acid fragment so that the function of one is affected by the other.

For example, a promoter (pro) is operably linked to a gene coding sequence (gene CDS) if it controls the transcription of the gene CDS (e.g., 5′-[pro]-[gene CDS]3′).

As used herein, the phrase “a variant rrn1-P2 promoter and 5′-UTR region” (abbreviated, “P2 promoter/5′-UTR region”) refers to the variant B. subtilis rrn1-P2 promoter/5′-aprE UTR region DNA sequence set forth in SEQ ID NO: 1.

As used herein, a DNA encoding a “wild-type B. subtilis aprE signal peptide sequence” may be abbreviated “aprE SS”, and comprises the nucleotide sequence of SEQ ID NO: 4.

As used herein, a “wild-type B. amyloliquefaciens BPN′ terminator (BPN′ term)” may be abbreviated “term”, and comprises the nucleotide sequence of SEQ ID NO: 6.

As used herein, a variant Bacillus gibsonii (BG46) subtilisin comprising the amino acid sequence of SEQ ID NO: 3 is abbreviated “BG46_variant 1”.

As used herein, a variant Bacillus gibsonii (BG46) subtilisin comprising the amino acid sequence of SEQ ID NO: 8 is abbreviated “BG46_variant 2”, which BG46_variant 2 was derived from the wild-type B. gibsonii (BG46) subtilisin reporter protein (SEQ ID NO: 2).

As used herein, “suitable regulatory sequences” refer to nucleotide sequences located upstream (5′ non-coding sequences), within, or downstream (3′ non-coding sequences) of a coding sequence, and which influence the transcription, RNA processing or stability, or translation of the associated coding sequence.

Regulatory sequences may include promoters, transcription leader sequences, RNA processing site, effector binding site and stem-loop structures.

As used herein, a “host cell” refers to a cell that has the capacity to act as a host or expression vehicle for a newly introduced DNA sequence. This, in certain embodiments of the disclosure, the host cells are Gram-positive cells (e.g., Bacillus sp.) and/or Gram-negative cells (e.g., E. coli).

As used herein, a “modified cell” refers to a recombinant cell that comprises at least one genetic modification which is not present in the parental, reference, or control cell from which the modified cell is derived.

As used herein, when the expression and/or production of a protein of interest (POI) in a recombinant (modified) cell is being compared to the expression and/or production of the same POI in an unmodified (control) cell, it will be understood that the modified and unmodified cells are grown/cultivated/fermented under the same conditions (e.g., the same conditions such as media, temperature, pH and the like).

As used herein, an “increased amount”, when used in phrases such as a “recombinant cell ‘expresses/produces an increased amount’ of a protein of interest relative to the unmodified (control) cell”, particularly refers to an “increased amount” of a protein of interest (POI) expressed/produced in by the recombinant cell, which “increased amount” is always relative to the unmodified (control) cells expressing/producing the same POI, wherein the modified and unmodified cells are grown/cultured/fermented under the same conditions.

As used herein, “increasing” protein production or “increased” protein production is meant an increased amount of protein produced (e.g., a protein of interest). The protein may be produced inside the host cell, or secreted (or transported) into the culture medium. In certain embodiments, the protein of interest is produced (secreted) into the culture medium. Increased protein production may be detected for example, as higher maximal level of protein or enzymatic activity (e.g., such as amylase activity), or total extracellular protein produced as compared to the parental host cell.

As used herein, the terms “modification” and “genetic modification” are used interchangeably and include: (a) the introduction, substitution, or removal of one or more nucleotides in a gene (or an ORF thereof), or the introduction, substitution, or removal of one or more nucleotides in a regulatory element required for the transcription or translation of the gene or ORF thereof, (b) a gene disruption, (c) a gene conversion, (d) a gene deletion, (e) the down-regulation of a gene, (f) specific mutagenesis and/or (g) random mutagenesis of any one or more the genes disclosed herein.

In certain aspects, genetic modifications particularly refer to the introduction, substitution, or removal of one or more nucleotides in a nucleic acid (DNA) sequence encoding pro-region (amino acid) sequence of the disclosure. For example, in certain embodiments, a DNA sequence encoding a native pro-region amino acid sequence set forth in SEQ ID NO: 15 is genetically modified as described herein.

As used herein, the term “introducing”, as used in phrases such as “introducing into a Gram-positive bacterial cell a ‘gene’, a ‘polynucleotide’, an ‘open reading frame’ (ORF), a ‘gene coding sequence, a ‘vector’, an ‘expression cassette’”, and the like, includes methods known in the art for introducing polynucleotides (DNA) into a cell, including, but not limited to protoplast fusion, natural or artificial transformation (e.g., calcium chloride, electroporation), transduction, transfection, conjugation and the like.

As used herein, “transformed” or “transformation” mean a cell has been transformed by use of recombinant DNA techniques. Transformation typically occurs by insertion of one or more nucleotide sequences (e.g., a polynucleotide, an ORF or gene) into a cell. The inserted nucleotide sequence may be a heterologous nucleotide sequence (i.e., a sequence that is not naturally occurring in cell that is to be transformed). Transformation therefore generally refers to introducing an exogenous DNA into a host cell so that the DNA is maintained as a chromosomal integrant or a self-replicating extra-chromosomal vector.

As used herein, “transforming DNA”, “transforming sequence”, and “DNA construct” refer to DNA that is used to introduce sequences into a host cell or organism. Transforming DNA is DNA used to introduce sequences into a host cell or organism. The DNA may be generated in vitro by PCR or any other suitable techniques. In some embodiments, the transforming DNA comprises an incoming sequence, while in other embodiments it further comprises an incoming sequence flanked by homology boxes. In yet a further embodiment, the transforming DNA comprises other non-homologous sequences, added to the ends (i.e., stuffer sequences or flanks). The ends can be closed such that the transforming DNA forms a closed circle, such as, for example, insertion into a vector.

As used herein, “disruption of a gene” or a “gene disruption”, are used interchangeably and refer broadly to any genetic modification that substantially prevents a host cell from producing a functional gene product (e.g., a protein). Thus, as used herein, a gene disruption includes, but is not limited to, frameshift mutations, premature stop codons (i.e., such that a functional protein is not made), substitutions eliminating or reducing activity of the protein internal deletions (such that a functional protein is not made), insertions disrupting the coding sequence, mutations removing the operable link between a native promoter required for transcription and the open reading frame, and the like.

As used herein “an incoming sequence” refers to a DNA sequence that is introduced into the bacterial cell chromosome. In some embodiments, the incoming sequence is part of a DNA construct. In other embodiments, the incoming sequence encodes one or more proteins of interest. In some embodiments, the incoming sequence comprises a sequence that may or may not already be present in the genome of the cell to be transformed (i.e., it may be either a homologous or heterologous sequence). In some embodiments, the incoming sequence encodes one or more proteins of interest, a gene, and/or a mutated or modified gene. In alternative embodiments, the incoming sequence encodes a functional wild-type gene or operon, a functional mutant gene or operon, or a nonfunctional gene or operon. In some embodiments, the non-functional sequence may be inserted into a gene to disrupt function of the gene. In another embodiment, the incoming sequence includes a selective marker. In a further embodiment the incoming sequence includes two homology boxes.

As used herein, “homology box” refers to a nucleic acid sequence, which is homologous to a sequence in the bacterial cell chromosome. More specifically, a homology box is an upstream or downstream region having between about 80 and 100% sequence identity, between about 90 and 100% sequence identity, or between about 95 and 100% sequence identity with the immediate flanking coding region of a gene or part of a gene to be deleted, disrupted, inactivated, down-regulated and the like, according to the invention. These sequences direct where in the bacterial cell chromosome a DNA construct is integrated and directs what part of the chromosome is replaced by the incoming sequence. While not meant to limit the present disclosure, a homology box may include about between 1 base pair (bp) to 200 kilobases (kb). Preferably, a homology box includes about between 1 bp and 10.0 kb; between 1 bp and 5.0 kb; between 1 bp and 2.5 kb; between 1 bp and 1.0 kb, and between 0.25 kb and 2.5 kb. A homology box may also include about 10.0 kb, 5.0 kb, 2.5 kb, 2.0 kb, 1.5 kb, 1.0 kb, 0.5 kb, 0.25 kb and 0.1 kb. In some embodiments, the 5′ and 3′ ends of a selective marker are flanked by a homology box wherein the homology box comprises nucleic acid sequences immediately flanking the coding region of the gene.

As used herein, a host cell “genome”, a bacterial (host) cell “genome”, or a Bacillus sp. (host) cell “genome” includes chromosomal and extrachromosomal genes.

As used herein, the terms “plasmid”, “vector” and “cassette” refer to extrachromosomal elements, often carrying genes which are typically not part of the central metabolism of the cell, and usually in the form of circular double-stranded DNA molecules. Such elements may be autonomously replicating sequences, genome integrating sequences, phage or nucleotide sequences, linear or circular, of a single-stranded or double-stranded DNA or RNA, derived from any source, in which a number of nucleotide sequences have been joined or recombined into a unique construction which is capable of introducing a promoter fragment and DNA sequence for a selected gene product along with appropriate 3′ untranslated sequence into a cell.

As used herein, the term “plasmid” refers to a circular double-stranded (ds) DNA construct used as a cloning vector, and which forms an extrachromosomal self-replicating genetic element in many bacteria and some eukaryotes. In some embodiments, plasmids become incorporated into the genome of the host cell. in some embodiments plasmids exist in a parental cell and are lost in the daughter cell.

A used herein, a “transformation cassette” refers to a specific vector comprising a gene (or ORF thereof), and having elements in addition to the foreign gene that facilitate transformation of a particular host cell.

As used herein, the term “vector” refers to any nucleic acid that can be replicated (propagated) in cells and can carry new genes or DNA segments into cells. Thus, the term refers to a nucleic acid construct designed for transfer between different host cells. Vectors include viruses, bacteriophage, pro-viruses, plasmids, phagemids, transposons, and artificial chromosomes such as YACs (yeast artificial chromosomes), BACs (bacterial artificial chromosomes), PLACs (plant artificial chromosomes), and the like, that are “episomes” (i.e., replicate autonomously or can integrate into a chromosome of a host organism).

An “expression vector” refers to a vector that has the ability to incorporate and express heterologous DNA in a cell. Many prokaryotic and eukaryotic expression vectors are commercially available and know to one skilled in the art. Selection of appropriate expression vectors is within the knowledge of one skilled in the art.

As used herein, the terms “expression cassette” and “expression vector” refer to a nucleic acid construct generated recombinantly or synthetically, with a series of specified nucleic acid elements that permit transcription of a particular nucleic acid in a target cell (i.e., these are vectors or vector elements, as described above). The recombinant expression cassette can be incorporated into a plasmid, chromosome, mitochondrial DNA, plastid DNA, virus, or nucleic acid fragment. Typically, the recombinant expression cassette portion of an expression vector includes, among other sequences, a nucleic acid sequence to be transcribed and a promoter. In some embodiments, DNA constructs also include a series of specified nucleic acid elements that permit transcription of a particular nucleic acid in a target cell. In certain embodiments, a DNA construct of the disclosure comprises a selective marker and an inactivating chromosomal or gene or DNA segment as defined herein.

As used herein, a “targeting vector” is a vector that includes polynucleotide sequences that are homologous to a region in the chromosome of a host cell into which the targeting vector is transformed and that can drive homologous recombination at that region. For example, targeting vectors find use in introducing mutations into the chromosome of a host cell through homologous recombination. In some embodiments, the targeting vector comprises other non-homologous sequences, e.g., added to the ends (i.e., stuffer sequences or flanking sequences). The ends can be closed such that the targeting vector forms a closed circle, such as, for example, insertion into a vector. For example, in certain embodiments, a parental B. licheniformis (host) cell is modified (e.g., transformed) by introducing therein one or more “targeting vectors”.

As used herein, the term “protein of interest” or “POI” refers to a polypeptide of interest that is desired to be expressed in a modified (recombinant) Gram-positive host cell, wherein the POI is preferably expressed at increased levels (i.e., relative to the “unmodified” (parental or control) cell). Thus, as used herein, a POI may be an enzyme, a substrate-binding protein, a surface-active protein, a structural protein, a receptor protein, and the like. In certain embodiments, a modified cell of the disclosure produces an increased amount of a heterologous protein of interest relative to the control cell. In particular embodiments, an increased amount of a protein of interest produced by a modified cell of the disclosure is at least a 0.5% increase, at least a 1.0% increase, at least a 5.0% increase, or a greater than 5.0% increase, relative to the control cell.

Similarly, as defined herein, a “gene of interest” or “GOI” refers a nucleic acid sequence (e.g., a polynucleotide, a gene or an ORF) which encodes a POI. A “gene of interest” encoding a “protein of interest” may be a naturally occurring gene, a mutated gene or a synthetic gene.

As used herein, the terms “polypeptide” and “protein” are used interchangeably, and refer to polymers of any length comprising amino acid residues linked by peptide bonds. The conventional one (1) letter or three (3) letter codes for amino acid residues are used herein. The polypeptide may be linear or branched, it may comprise modified amino acids, and it may be interrupted by non-amino acids. The term polypeptide also encompasses an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art.

In certain embodiments, a gene of the instant disclosure encodes a commercially relevant industrial protein of interest, such as an enzyme (e.g., a acetyl esterases, aminopeptidases, amylases, arabinases, arabinofuranosidases, carbonic anhydrases, carboxypeptidases, catalases, cellulases, chitinases, chymosins, cutinases, deoxyribonucleases, epimerases, esterases, α-galactosidases, β-galactosidases, α-glucanases, glucan lysases, endo-β-glucanases, glucoamylases, glucose oxidases, α-glucosidases, β-glucosidases, glucuronidases, glycosyl hydrolases, hemicellulases, hexose oxidases, hydrolases, invertases, isomerases, laccases, lipases, lyases, mannosidases, oxidases, oxidoreductases, pectate lyases, pectin acetyl esterases, pectin depolymerases, pectin methyl esterases, pectinolytic enzymes, perhydrolases, polyol oxidases, peroxidases, phenoloxidases, phytases, polygalacturonases, proteases, peptidases, rhamno-galacturonases, ribonucleases, transferases, transport proteins, transglutaminases, xylanases, hexose oxidases, and combinations thereof).

As used herein, a “variant” polypeptide refers to a polypeptide that is derived from a parent (or reference) polypeptide by the substitution, addition, or deletion of one or more amino acids, typically by recombinant DNA techniques. Variant polypeptides may differ from a parent polypeptide by a small number of amino acid residues and may be defined by their level of primary amino acid sequence homology/identity with a parent (reference) polypeptide.

In certain one or more embodiments, variant polypeptides have at least about 40% to about 70%, at least 75%, at least 80%, at least 85%, at least 90%, at least 91%, at least 92%, at least 93%, at least 94%, at least 95%, at least 96%, at least 97%, at least 98%, or even at least 99% amino acid sequence identity with a parent (reference) polypeptide sequence.

As used herein, a “variant” polynucleotide refers to a polynucleotide having a specified degree of sequence homology/identity with a parent (or reference) polynucleotide, or hybridizes with a parent polynucleotide (or a complement thereof) under stringent hybridization conditions. In certain one or more embodiments, variant polynucleotides of the disclosure comprise at least about 40% to at least about 100% nucleotide sequence identity with a parent (reference) polynucleotide sequence. In certain other embodiments, a variant polynucleotide comprises at least about 40%, at least about 45%, at least about 50%, at least about 55%, at least about 60%, at least about 65%, at least about 70%, at least about 75%, at least about 80%, at least about 85%, at least about 90%, at least 91%, at least 92%, at least 93%, at least 94%, at least 95%, at least 96%, at least 97%, at least 98%, or at least 99% nucleotide sequence identity with a parent (reference) polynucleotide sequence.

As used herein, a “mutation” refers to any change or alteration in a nucleic acid sequence. Several types of mutations exist, including point mutations, deletion mutations, silent mutations, frame shift mutations, splicing mutations and the like. Mutations may be performed specifically (e.g., via site directed mutagenesis) or randomly (e.g., via chemical agents, passage through repair minus bacterial strains).

As used herein, in the context of a polypeptide or a sequence thereof, the term “substitution” means the replacement (i.e., substitution) of one amino acid with another amino acid.

As used herein, the term “homology” relates to homologous polynucleotides or polypeptides. If two or more polynucleotides or two or more polypeptides are homologous, this means that the homologous polynucleotides or polypeptides have a “degree of identity” of at least 60%, more preferably at least 70%, even more preferably at least 85%, still more preferably at least 90%, more preferably at least 95%, and most preferably at least 98%. Whether two polynucleotide or polypeptide sequences have a sufficiently high degree of identity to be homologous as defined herein, can suitably be investigated by aligning the two sequences using a computer program known in the art, such as “GAP” provided in the GCG program package (Program Manual for the Wisconsin Package, Version 8, August 1994, Genetics Computer Group, 575 Science Drive, Madison, Wisconsin, USA 53711) (Needleman and Wunsch, (1970). Using GAP with the following settings for DNA sequence comparison: GAP creation penalty of 5.0 and GAP extension penalty of 0.3.

As used herein, the term “percent (%) identity” refers to the level of nucleic acid or amino acid sequence identity between the nucleic acid sequences that encode a polypeptide or the polypeptide's amino acid sequences, when aligned using a sequence alignment program.

As used herein, “specific productivity” is total amount of protein produced per cell per time over a given time period.

As used herein, the terms “purified”, “isolated” or “enriched” are meant that a biomolecule (e.g., a polypeptide or polynucleotide) is altered from its natural state by virtue of separating it from some, or all of, the naturally occurring constituents with which it is associated in nature. Such isolation or purification may be accomplished by art-recognized separation techniques such as ion exchange chromatography, affinity chromatography, hydrophobic separation, dialysis, protease treatment, ammonium sulphate precipitation or other protein salt precipitation, centrifugation, size exclusion chromatography, filtration, microfiltration, gel electrophoresis or separation on a gradient to remove whole cells, cell debris, impurities, extraneous proteins, or enzymes undesired in the final composition. It is further possible to then add constituents to a purified or isolated biomolecule composition which provide additional benefits, for example, activating agents, anti-inhibition agents, desirable ions, compounds to control pH or other enzymes or chemicals.

### II. MUTANT PRO-SEQUENCES FOR ENHANCED PROTEIN PRODUCTION

As generally described herein and set forth below in the Examples, Applicant has designed and constructed a site evaluation library (SEL) to test/screen certain genetic modifications (mutations) for enhanced productivity of recombinant proteins. In particular, as set forth in the Examples, recombinant proteins (enzymes) were used as reporters to monitor protein expression in recombinant Bacillus strains of the disclosure. Certain pro-region sequences suitable for use in the expression/production of heterologous (recombinant) mature proteins have been described. For example, PCT Publication No. WO2008/112258 and PCT Publication No. 2010/123754 describe the pro-sequences of the B. clausii alkaline precursor protease and the B. lentus subtilisin precursor protease, wherein certain mutant pro-sequences resulted in enhanced productivity of the mature protease. PCT Publication No. WO1993/20214 describes certain AprE signal (pre) and pro-region sequences for the expression of a gram-negative lipase in B. subtilis.

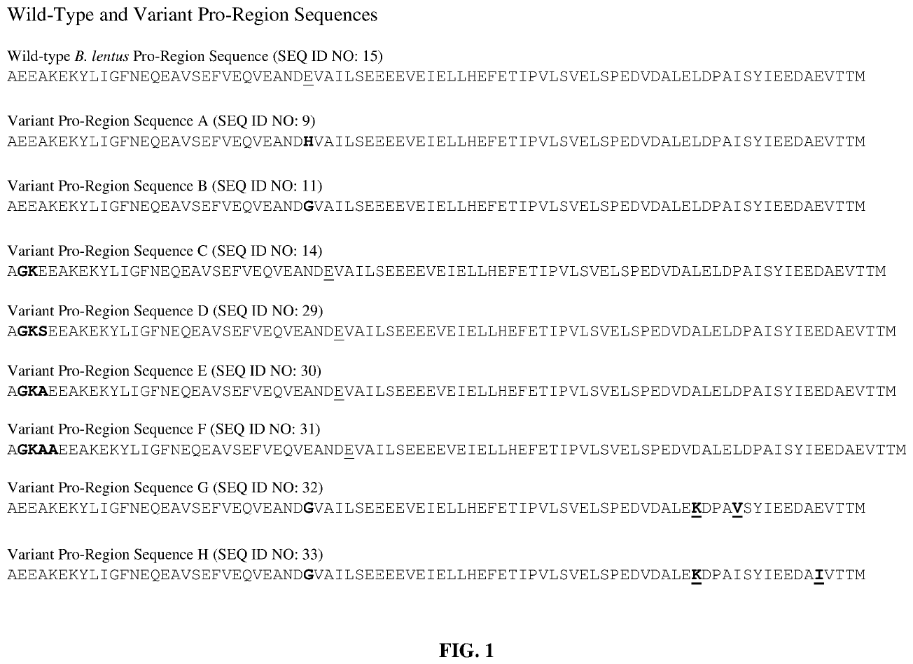

More particularly, as described in Examples below, genetic modifications (SEL) were performed on the pro-region of expression constructs encoding exemplary (subtilisin) reporter proteins. In particular, as generally set forth in the Examples (see, FIG. 1-FIG. 3) an SEL was performed on pro-region sequences derived from a wild-type (reference) pro-sequence (SEQ ID NO: 15) to screen mutations for enhanced productivity of the mature subtilisin reporter protein.

As described in Example 1, the amino acid sequence encoding the variant pro-region sequence A (30H, SEQ ID NO: 9; FIG. 1) was used as template to create site evaluation libraries (SEL) and developed as 4.4 kb fragments, wherein the linear DNA of the expression cassettes were used to transform competent Bacillus cells. As described in Example 1, sequence analysis was performed to determine unique pro-region sequence A variants and/or pro-region sequence B variants from the SEL, wherein each of the 84 amino acid positions of the variant pro-region sequence A (SEQ ID NO: 9) were altered (substituted) to all other nineteen (19) naturally occurring amino acid residues. In particular, TABLE 1 (Example 1) shows the results of the reporter protein productivity (performance index (PI) values) after seventy-two (72) hours as compared to the reference construct in which the reporter protein was expressed with the WT PRO sequence (SEQ ID NO: 15). For example, as shown in TABLE 1, the PI of pro-region sequence A (E30H, SEQ ID NO: 9) was about 50% increased relative to the PI of the WT PRO sequence (30E; SEQ ID NO: 15). Likewise, as indicated in TABLE 1, mutations at any one of amino acid positions 1, 2, 3, 4, 6, 14, 19, 20, 23, 36, 37, 38, 39, 42, 43, 44, 49, 50, 64, 65, 67, 68, 71, 83 or 84 in combination with position 30 demonstrated enhanced reporter protein productivity at 72 hours relative to the reference pro-region sequence (SEQ ID NO: 15). Additionally, about 80% of the selected mutant combinations had an extra positive charge (see, TABLE 1, A Charge).

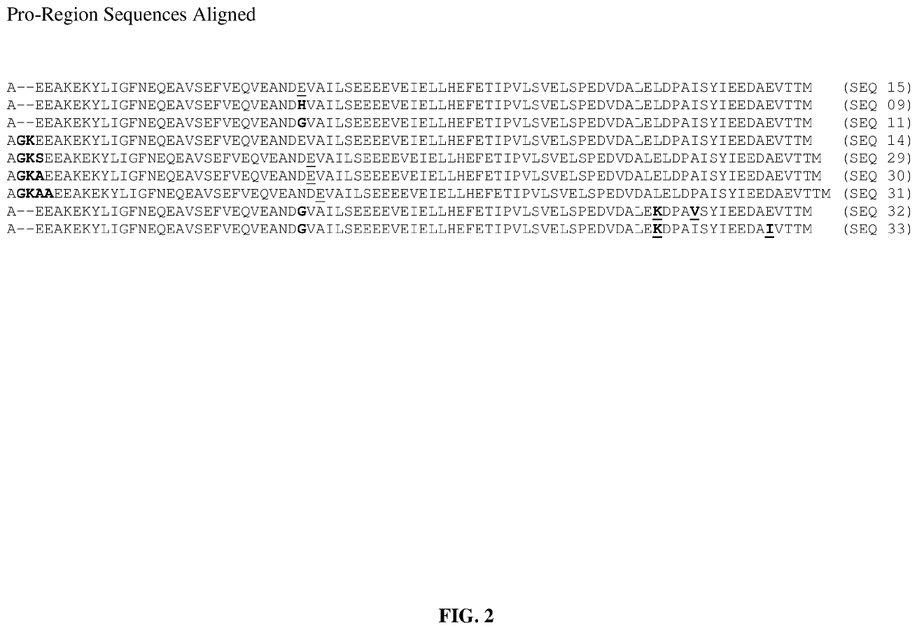

As further described in Example 3, genetic modifications (SEL) were performed on the pro-region of expression constructs encoding an exemplary (subtilisin) reporter protein (BG46_variant 2). More particularly, the amino acid sequence encoding variant pro-region sequence B (30G, SEQ ID NO: 11; FIG. 1) was used as template to develop linear DNA expression cassettes comprising one or more pro-regions of the disclosure. In particular, the 5′ sequence of the seventy-eight (78) amino acid wild-type AprE pro-peptide sequence (SEQ ID NO: 12) was used to exchange the first three (3) amino acid residues (positions 1-3, “AGK”; SEQ ID NO: 12) with the first amino acid residue which is alanine (A), resulting in variant pro-region sequence C (SEQ ID NO: 14; GK insertion, FIG. 2). For example, as shown in TABLE 1 (Example1), a mutant of the pro-region variant A sequence (SEQ ID NO: 9; TABLE 1, mutant “E30G”) showed high expression of the reporter protein after 72 hours of growth (PI 2.3) as compared to the wild-type pro-region (TABLE 1, PI 1.0) and the pro-region variant A sequence (SEQ ID NO: 11; TABLE 1, mutant “E30H”, PI 1.5).

Example 4 of the disclosure further describes the preparation of combinatorial libraries based on the reference pro-region sequence (SEQ ID NO: 15) in which the amino acids glycine (G) and lysine (K) were inserted (“GK”). More particularly, as described in Example 3, the amino acid residues GK were inserted at the N-terminus of the wild-type pro-region sequence (SEQ ID NO: 15), resulting in the pro-region variant C amino acid sequence set forth in SEQ ID NO: 14, comprising 86 amino acid positions shown in the FIG. 2 alignment. For example, TABLE 2 (Example 4) presents combinations of pro-region mutations that showed increased productivity (PI) of the BG46_variant 2 reporter protein, wherein the change in charge (A Charge) was generally between +1 and +4 (TABLE 2). In certain aspects, Applicant notes that such positive charges are suitable as combinations in the loop region of the pro-region sequence (e.g., such as residue positions 66-73 of reference SEQ ID NO:15).

As shown in TABLE 2, the PI values of the SEL pro-region variant C sequences (derived from SEQ ID NO: 14) are compared to the PI value of the wild-type (WT) pro-region sequence (SEQ ID NO: 15), wherein the amino acid position numbering (TABLE 2, 1^{st} column) is in comparison with WT pro-region sequence (i.e., without “AGK” insertion). Thus, as described in Example 4, the pro-region variant C sequences set forth in TABLE 2 showed high expression of the reporter protein after 72 hours of growth (PI values between about 1.9 and 2.3) as compared to the wild-type pro-region (TABLE 2, PI 1.0) and the pro-region variant B sequence (mutant “E30G”, PI 1.7).

As generally described in Example 5, the BG46_variant 2 subtilisin was used as a reporter protein to monitor expression as in B. licheniformis strains. More particularly, DNA encoding a variant pro-region sequence of the disclosure was operably linked to a DNA sequence encoding the mature BG46_variant 2 protein, with strains constructed as generally described in Example 5. For example, the PI values B. licheniformis strains comprising a variant pro-region sequence are shown in TABLE 3 relative to the control pro-region sequence B comprising the E30G mutation (SEQ ID NO: 11), wherein variant pro-region sequences showed high expression of the reporter protein after 72 hours of growth as compared to the control pro-region sequence (E30G).

As further described in Example 6, additional N-terminus modifications of the variant pro-region sequence C (SEQ ID NO: 14) were performed. For instance, the variant pro-region sequence C (having 86 amino acid positions) was further modified to introduce (insert) additional amino acids such as alanine (A) or serine (S) after the lysine (K) in position three (3). In particular, as described in Example 6 (and shown in FIG. 1-FIG. 3), variant pro-region sequences D (SEQ ID NO: 29, GKS insertion), E (SEQ ID NO: 30, GKA insertion) and F (GKAA insertion; SEQ ID NO: 31) were constructed, wherein the performance index (PI) values of the pro-region variant sequences D, E and F (TABLE 4) are compared to the variant pro-region sequence C (SEQ ID NO: 14, “GK” insertion). As presented in TABLE 4, the PI index of the three N-terminal pro-region mutants (GKA, GKS, GKAA) demonstrated higher expression of the reporter protein BG46_variant 2 subtilisin (SEQ ID NO: 8) after 72 hours of growth as compared to pro-region sequence C (GK; SEQ ID NO: 14).

Likewise, certain C-terminal modifications of the pro-region sequence were performed as described in Example 7. In particular, the variant pro-region sequence B (SEQ ID NO: 11) was further engineered to substitute the leucine (L) residue at position sixty-eight (68) with lysine (K) and substitute the isoleucine (I) at position seventy-two (72) with valine (V) to generate variant pro-region sequence G (SEQ ID NO: 32), or engineered to substitute the leucine (L) residue at position sixty-eight (68) with lysine (K) and substitute the glutamic acid (E) at position seventy-two (80) with an isoleucine (I) to generate variant pro-region sequence F (SEQ ID NO: 33), as shown in FIG. 1-FIG. 3. As presented in TABLE 5, the PI values of the variant pro-region sequences G (SEQ ID NO: 32) and H (SEQ ID NO: 33) were increased as compared to the reference (control) variant pro-region B sequence (SEQ ID NO: 11).

Thus, certain embodiments of the disclosure provide, inter alia, recombinant polynucleotides encoding novel pro-region sequences, expression cassettes comprising DNA sequences encoding novel pro-region sequences operably linked to DNA sequences encoding mature proteins of interest, modified Gram-positive bacterial cells/strains expressing polynucleotides encoding precursor proteins comprising novel pro-region sequences, and the like. In certain aspects, novel variant (mutant) pro-region sequences of the disclosure comprise an amino acid sequence derived from a wild-type B. lentus pro-sequence of SEQ ID NO: 15.

As used herein, the phrase “variant pro-region” refers to a polypeptide sequence derived from a reference pro-region sequence by the substitution, addition, or deletion of one or more amino acids, typically by recombinant DNA techniques. Variant pro-region (amino acid) sequences may differ from a reference (parent) pro-region sequence by a small number of amino acid residues and may be defined by their level of primary amino acid sequence homology/identity with a reference (parent) pro-region sequence.

As set forth above, the term “identical” in the context of two polynucleotide or polypeptide sequences refers to the nucleotides or amino acids in the two sequences that are the same when aligned for maximum correspondence, as measured using sequence comparison or analysis algorithms described below and known in the art. The phrase “percent (%) identity” (abbreviated, “PID”) refers to polynucleotide (nucleic acid) or polypeptide (amino acid) sequence identity. Percent identity may be determined using standard techniques known in the art. In certain aspects, the percent amino acid identity shared by sequences of interest can be determined by aligning the sequences to directly compare the sequence information, e.g., by using an alignment program/algorithm such as BLAST, MUSCLE, or CLUSTAL. For example, the BLAST algorithm has been described in Altschul et al. (1990) and Karlin et al. (1993). In particular, a percent (%) amino acid sequence identity value is determined by the number of matching identical residues divided by the total number of residues of the “reference” sequence including any gaps created by the program for optimal/maximum alignment. BLAST algorithms refer to the “reference” sequence as the “query” sequence.

As used herein, “homologous pro-regions” refer to pro-regions that have distinct similarity in primary, secondary, and/or tertiary structure. Protein homology can refer to the similarity in linear amino acid sequence when proteins are aligned. Homology can be determined by amino acid sequence alignment, e.g., using a program such as BLAST, MUSCLE, or CLUSTAL. Homologous search of protein sequences can be done using BLASTP and PSI-BLAST from NCBI BLAST with threshold (E-value cut-off) at 0.001 (e.g., see Altschul et al., 1997).

The BLAST program uses several search parameters, most of which are set to the default values. The NCBI BLAST algorithm finds the most relevant sequences in terms of biological similarity but is not recommended for query sequences of less than 20 residues (Altschul et al., 1997 and Schaffer et al., 2001). Exemplary default BLAST parameters for a nucleic acid sequence searches include: Neighboring words threshold=11; E-value cutoff=10; Scoring Matrix=NUC.3.1 (match=1, mismatch=−3); Gap Opening=5; and Gap Extension=2. Exemplary default BLAST parameters for amino acid sequence searches include: Word size=3; E-value cutoff=10; Scoring Matrix=BLOSUM62; Gap Opening=11; and Gap extension=1. Using this information, protein sequences can be grouped and/or a phylogenetic tree built therefrom. Amino acid sequences can be entered in a program such as the Vector NTI Advance suite and a Guide Tree can be created using the Neighbor Joining (NJ) method (Saitou and Nei, 1987). The tree construction can be calculated using Kimura's correction for sequence distance and ignoring positions with gaps. A program such as AlignX can display the calculated distance values in parenthesis following the molecule name displayed on the phylogenetic tree.

Understanding the homology between molecules can reveal the evolutionary history of the molecules as well as information about their function; if a newly sequenced protein is homologous to an already characterized protein, there is a strong indication of the new protein's biochemical function. Two molecules are said to be homologous if they have been derived from a common ancestor. Homologous molecules, or homologs, can be divided into two classes, paralogs and orthologs. Paralogs are homologs that are present within one species. Paralogs often differ in their detailed biochemical functions. Orthologs are homologs that are present within different species and have very similar or identical functions. A protein superfamily is the largest grouping (clade) of proteins for which common ancestry can be inferred. Usually this common ancestry is based on sequence alignment and mechanistic similarity.

The CLUSTAL W algorithm is another example of a sequence alignment algorithm (Thompson et al., 1994). Default parameters for the CLUSTAL W algorithm include: Gap opening penalty=10.0; Gap extension penalty=0.05; Protein weight matrix=BLOSUM series; DNA weight matrix=IUB; Delay divergent sequences %=40; Gap separation distance=8; DNA transitions weight=0.50; List hydrophilic residues=GPSNDQEKR; Use negative matrix=OFF; Toggle Residue specific penalties=ON; Toggle hydrophilic penalties=ON; and Toggle end gap separation penalty=OFF. In CLUSTAL algorithms, deletions occurring at either terminus are included. For example, a variant with a five amino acid deletion at either terminus (or within the polypeptide) of a polypeptide of 500 amino acids would have a percent sequence identity of 99% (495/500 identical residues×100) relative to the “reference” polypeptide. Such a variant would be encompassed by a variant having “at least 99% sequence identity” to the polypeptide.

In certain embodiments, variant pro-region sequences have at least about 40% to about 70%, at least 75%, at least 80%, at least 85%, at least 90%, at least 91%, at least 92%, at least 93%, at least 94%, at least 95%, at least 96%, at least 97%, at least 98%, or even at least 99% amino acid sequence identity with a reference (parent) pro-region sequence of the disclosure. In certain embodiments, a pro-region sequence is derived from an amino acid sequence comprising homology to SEQ ID NO: 15, SEQ ID NO: 9, SEQ ID NO: 11, SEQ ID NO: 14, SEQ ID NO: 29, SEQ ID NO: 30, SEQ ID NO: 31, SEQ ID NO: 32 and/or SEQ ID NO: 33. Thus, in certain aspects, amino acid modifications of the one or more pro-region variants described herein are numbered by reference to a pro-region amino acid sequence of SEQ ID NO: 15, SEQ ID NO: 9, SEQ ID NO: 11, SEQ ID NO: 14, SEQ ID NO: 29, SEQ ID NO: 30, SEQ ID NO: 31, SEQ ID NO: 32 and/or SEQ ID NO: 33.

In certain aspects, novel (variant) pro-region sequences are derived from a parent (reference) pro-region sequence having at least 80%, 85%, 86%, 87%, 88%, 89%, 90%, 91%, 92%, 93%, 94%, 95%, 96%, 97%, 98%, 99%, or 100% amino acid sequence identity to the amino acid sequence of SEQ ID NO: 15. In certain embodiments, variant pro-region sequences comprise a mutation at amino acid (residue) position 30, and one or more amino acid residue positions selected from the group consisting of positions 1, 2, 3, 4, 6, 14, 16, 19, 20, 23, 36, 37, 38, 39, 42, 43, 44, 49, 50, 64, 65, 67, 68, 71, 79, 83 and 84, according to SEQ ID NO: 15 amino acid (position) numbering.

In other embodiments, variant pro-region sequences of the disclosure comprise amino acid insertions of glycine (G) at position 2 and lysine (K) at position 3, and an amino acid substitution at one or more positions selected from positions 1, 32, 38, 46, 66, 67, 70 and 73, wherein the amino acid positions are numbered according to SEQ ID NO: 14. In certain embodiments, novel (variant) pro-region sequences are derived from a reference pro-region sequence comprising at least 80%, 85%, 86%, 87%, 88%, 89%, 90%, 91%, 92%, 93%, 94%, 95%, 96%, 97%, 98%, 99%, or 100% amino acid sequence identity to the amino acid sequence of SEQ ID NO: 14.

In other embodiments, variant pro-region sequences of the disclosure comprise amino acid insertions of glycine (G) at position 2, lysine (K) at position 3, and serine (S) at position 4, wherein the amino acid positions are numbered according to SEQ ID NO: 29. In certain embodiments, novel (variant) pro-region sequences are derived from a reference pro-region sequence comprising at least 80%, 85%, 86%, 87%, 88%, 89%, 90%, 91%, 92%, 93%, 94%, 95%, 96%, 97%, 98%, 99%, or 100% amino acid sequence identity to the amino acid sequence of SEQ ID NO: 29.

In other embodiments, variant pro-region sequences of the disclosure comprise amino acid insertions of glycine (G) at position 2, lysine (K) at position 3, and alanine (A) at position 4, wherein the amino acid positions are numbered according to SEQ ID NO: 30. In certain embodiments, variant pro-region sequences are derived from a reference pro-region sequence comprising at least 80%, 85%, 86%, 87%, 88%, 89%, 90%, 91%, 92%, 93%, 94%, 95%, 96%, 97%, 98%, 99%, or 100% amino acid sequence identity to the amino acid sequence of SEQ ID NO: 30.

In other embodiments, variant pro-region sequences of the disclosure comprise amino acid insertions of glycine (G) at position 2, lysine (K) at position 3, alanine (A) at position 4, and alanine (A) at position 5, wherein the amino acid positions are numbered according to SEQ ID NO: 31. In certain embodiments, variant pro-region sequences are derived from a reference pro-region sequence comprising at least 80%, 85%, 86%, 87%, 88%, 89%, 90%, 91%, 92%, 93%, 94%, 95%, 96%, 97%, 98%, 99%, or 100% amino acid sequence identity to the amino acid sequence of SEQ ID NO: 31.

In certain other embodiments, variant pro-region sequences are derived from a parent (reference) pro-region sequence having at least 80%, 85%, 86%, 87%, 88%, 89%, 90%, 91%, 92%, 93%, 94%, 95%, 96%, 97%, 98%, 99%, or 100% amino acid sequence identity to the amino acid sequence of SEQ ID NO: 11 (G30; pro-region B sequence).

In certain embodiments, variant pro-region sequences comprise at least one amino acid substitution at a position selected from position 68, position 72 and position 80, wherein the amino acid positions are numbered according to SEQ ID NO: 9, SEQ ID NO: 11, SEQ ID NO: 15, SEQ ID NO: 32, or SEQ ID NO: 33.

In certain other embodiments, variant pro-region sequences comprise a mutation at amino acid (residue) position 30, and one or more amino acid residue positions selected from the group consisting of positions 1, 2, 3, 4, 6, 14, 16, 19, 20, 23, 36, 37, 38, 39, 42, 43, 44, 49, 50, 64, 65, 67, 68, 71, 79, 83 and 84, according to SEQ ID NO: 9, SEQ ID NO: 11, SEQ ID NO: 15, SEQ ID NO: 32, or SEQ ID NO: 33 amino acid (position) numbering.

In certain embodiments, variant pro-region sequences are designed, engineered, constructed and the like, such that the net charge of the pro-region sequence is a positive charge (e.g., +1 net charge) relative to a reference (parent) pro-region sequence such as the reference (parent) sequences of SEQ ID NO: 9, SEQ ID NO: 11, SEQ ID NO: 14, SEQ ID NO: 15, SEQ ID NO: 29, SEQ ID NO: 30, SEQ ID NO: 31, SEQ ID NO: 32 and SEQ ID NO: 33. In certain embodiments, variant pro-region sequences of the disclosure are designed, engineered, constructed and the like, such that the net charge of the pro-region sequence is at least positive 1 (+1) to about positive 4 (+4). In other one or more embodiments, variant pro-region sequences of the disclosure comprise one or more amino acid modifications set forth in TABLE 1, TABLE 2, TABLE 3, TABLE 4 and/or TABLE 5.

### III. RECOMBINANT POLYNUCLEOTIDES AND MOLECULAR BIOLOGY

As generally set forth above, certain embodiments of the disclosure are related to novel mutant pro-region sequences. In related aspects, the disclosure provides recombinant polynucleotides comprising one or more mutant pro-region nucleic acid (DNA) sequences. Thus, certain embodiments are related to recombinant polynucleotides (e.g., vectors, plasmids, expression cassettes, etc.), recombinant Gram-positive bacterial cells/strains expressing proteins of interest and the like. In certain aspects, the disclosure provides polynucleotide constructs suitable for introducing into recombinant Gram-positive bacterial cells (strains) for the enhanced production of proteins of interest. In certain aspects, a polynucleotide construct of the disclosure is referred to as an expression cassette, wherein the cassette comprises, in the 5′ to 3′ direction and in operable combination, at least an upstream (5′) a pro-region DNA sequence linked to a downstream (3′) gene CDS encoding a mature protein on interest (POI).

For example, one or more nucleic acid sequences described herein can be generated by using any suitable synthesis, manipulation, and/or isolation techniques, or combinations thereof. For example, one or more polynucleotides described herein may be produced using standard nucleic acid synthesis techniques, such as solid-phase synthesis techniques that are well-known to those skilled in the art. In such techniques, fragments of up to fifty (50) or more nucleotide bases are typically synthesized, then joined (e.g., by enzymatic or chemical ligation methods) to form essentially any desired continuous nucleic acid sequence. The synthesis of the one or more polynucleotide described herein can be also facilitated by any suitable method known in the art, including but not limited to chemical synthesis using the classical phosphoramidite method (e.g., Beaucage and Caruthers, 1981) or the method described by Matthes et al. (1984) as is typically practiced in automated synthetic methods. One or more polynucleotides described herein can also be produced by using an automatic DNA synthesizer. Customized nucleic acids can be ordered from a variety of commercial sources (e.g., ATUM (DNA 2.0), Newark, CA, USA; Life Tech (GeneArt), Carlsbad, CA, USA; GenScript, Ontario, Canada; Base Clear B. V., Leiden, Netherlands; Integrated DNA Technologies, Skokie, IL, USA; Ginkgo Bioworks (Gen9), Boston, MA, USA; and Twist Bioscience, San Francisco, CA, USA). Other techniques for synthesizing nucleic acids and related principles are described and known in the art.

Recombinant DNA techniques useful in modification of nucleic acids are well known in the art, such as, for example, restriction endonuclease digestion, ligation, reverse transcription and cDNA production, and polymerase chain reaction (e.g., PCR). One or more polynucleotides described herein may also be obtained by screening cDNA libraries using one or more oligonucleotide probes that can hybridize to or PCR-amplify polynucleotides which encode one or more variants described herein. Procedures for screening and isolating cDNA clones and PCR amplification procedures are well known to those of skill in the art and described in standard references known to those skilled in the art. One or more polynucleotides described herein can be obtained by altering a naturally occurring polynucleotide backbone (e.g., that encodes one or more variant pro-region sequences described herein) by, for example, a known mutagenesis procedure (e.g., site-directed mutagenesis, site saturation mutagenesis, and in vitro recombination). A variety of methods are known in the art that are suitable for generating modified polynucleotides described herein that encode one or more variants described herein, including, but not limited to, for example, site-saturation mutagenesis, scanning mutagenesis, insertional mutagenesis, deletion mutagenesis, random mutagenesis, site-directed mutagenesis, and directed-evolution, as well as various other recombinatorial approaches.

As generally set forth above and further described below in the Examples, certain embodiments of the disclosure are related to recombinant (modified) Gram-positive cells capable of producing increased amounts of heterologous proteins of interest. Certain embodiments are therefore related to methods for constructing such recombinant Gram-positive cells having increased protein production capabilities. In certain embodiments, one or more expression cassettes encoding a protein of interest are introduced into Gram-positive cells of the disclosure. In exemplary embodiments, the cassettes are integrated into the genome of the cell. Thus, certain embodiments are related to nucleic acid molecules, polynucleotides (e.g., vectors, plasmids, expression cassettes), regulatory elements, and the like, suitable for use in constructing recombinant (modified) Gram-positive host cells.

Accordingly, as presented in the Examples and generally described herein, recombinant cells of the disclosure may be constructed by one of skill using standard and routine recombinant DNA and molecular cloning techniques well known in the art. Methods for genetic modification include, but are not limited to, (a) the introduction, substitution, or removal of one or more nucleotides in a gene, or the introduction, substitution, or removal of one or more nucleotides in a regulatory element required for the transcription or translation of the gene, (b) a gene disruption, (c) a gene conversion, (d) a gene deletion, (e) a gene down-regulation, (f) site specific mutagenesis and/or (g) random mutagenesis.

In certain embodiments, modified cells of the disclosure may be constructed by reducing or eliminating the expression of a gene, using methods well known in the art, for example, insertions, disruptions, replacements, or deletions. The portion of the gene to be modified or inactivated may be, for example, the coding region or a regulatory element required for expression of the coding region.

An example of such a regulatory or control sequence may be a promoter sequence or a functional part thereof, (i.e., a part which is sufficient for affecting expression of the nucleic acid sequence). Other control sequences for modification include, but are not limited to, a leader sequence, a pro-peptide sequence, a signal sequence, a transcription terminator, a transcriptional activator and the like.

In certain other embodiments a modified cell is constructed by gene deletion to eliminate or reduce the expression of the gene. Gene deletion techniques enable the partial or complete removal of the gene(s), thereby eliminating their expression, or expressing a non-functional (or reduced activity) protein product. In such methods, the deletion of the gene(s) may be accomplished by homologous recombination using a plasmid that has been constructed to contiguously contain the 5′ and 3′ regions flanking the gene. The contiguous 5′ and 3′ regions may be introduced into a cell, for example, on a temperature-sensitive plasmid in association with a second selectable marker at a permissive temperature to allow the plasmid to become established in the cell. The cell is then shifted to a non-permissive temperature to select for cells that have the plasmid integrated into the chromosome at one of the homologous flanking regions. Selection for integration of the plasmid is affected by selection for the second selectable marker. After integration, a recombination event at the second homologous flanking region is stimulated by shifting the cells to the permissive temperature for several generations without selection. The cells are plated to obtain single colonies and the colonies are examined for loss of both selectable markers. Thus, a person of skill in the art may readily identify nucleotide regions in the gene's coding sequence and/or the gene's non-coding sequence suitable for complete or partial deletion.

In other embodiments, a modified cell is constructed by introducing, substituting, or removing one or more nucleotides in the gene or a regulatory element required for the transcription or translation thereof. For example, nucleotides may be inserted or removed so as to result in the introduction of a stop codon, the removal of the start codon, or a frame-shift of the open reading frame. Such a modification may be accomplished by site-directed mutagenesis or PCR generated mutagenesis in accordance with methods known in the art. Thus, in certain embodiments, a gene of the disclosure is inactivated by complete or partial deletion.

In another embodiment, a modified cell is constructed by the process of gene conversion. For example, in the gene conversion method, a nucleic acid sequence corresponding to the gene(s) is mutagenized in vitro to produce a defective nucleic acid sequence, which is then transformed into the parental cell to produce a defective gene. By homologous recombination, the defective nucleic acid sequence replaces the endogenous gene. It may be desirable that the defective gene or gene fragment also encodes a marker which may be used for selection of transformants containing the defective gene. For example, the defective gene may be introduced on a non-replicating or temperature-sensitive plasmid in association with a selectable marker. Selection for integration of the plasmid is affected by selection for the marker under conditions not permitting plasmid replication. Selection for a second recombination event leading to gene replacement is affected by examination of colonies for loss of the selectable marker and acquisition of the mutated gene. Alternatively, the defective nucleic acid sequence may contain an insertion, substitution, or deletion of one or more nucleotides of the gene, as described below.

In other embodiments, a modified cell is constructed by established anti-sense techniques using a nucleotide sequence complementary to the nucleic acid sequence of the gene. More specifically, expression of the gene by a Gram-positive cell may be reduced (down-regulated) or eliminated by introducing a nucleotide sequence complementary to the nucleic acid sequence of the gene, which may be transcribed in the cell and is capable of hybridizing to the mRNA produced in the cell. Under conditions allowing the complementary anti-sense nucleotide sequence to hybridize to the mRNA, the amount of protein translated is thus reduced or eliminated. Such anti-sense methods include, but are not limited to RNA interference (RNAi), small interfering RNA (siRNA), microRNA (miRNA), antisense oligonucleotides, and the like, all of which are well known to the skilled artisan.

In other embodiments, a modified cell is produced/constructed via CRISPR-Cas9 editing. For example, a gene encoding a protein of interest can be edited or disrupted (or deleted or down-regulated) by means of nucleic acid guided endonucleases, that find their target DNA by binding either a guide RNA (e.g., Cas9) and Cpf1 or a guide DNA (e.g., NgAgo), which recruits the endonuclease to the target sequence on the DNA, wherein the endonuclease can generate a single or double stranded break in the DNA. This targeted DNA break becomes a substrate for DNA repair, and can recombine with a provided editing template to disrupt or delete the gene. For example, the gene encoding the nucleic acid guided endonuclease (for this purpose Cas9 from S. pyogenes) or a codon optimized gene encoding the Cas9 nuclease is operably linked to a promoter active in the Gram-positive cell and a terminator active in Gram-positive cells, thereby creating a Gram-positive cell Cas9 expression cassette. Likewise, one or more target sites unique to the gene of interest are readily identified by a person skilled in the art. For example, to build a DNA construct encoding a gRNA-directed to a target site within the gene of interest, the variable targeting domain (VT) will comprise nucleotides of the target site which are 5′ of the (PAM) proto-spacer adjacent motif (TGG), which nucleotides are fused to DNA encoding the Cas9 endonuclease recognition domain for S. pyogenes Cas9 (CER). The combination of the DNA encoding a VT domain and the DNA encoding the CER domain thereby generate a DNA encoding a gRNA. Thus, a Gram-positive expression cassette for the gRNA is created by operably linking the DNA encoding the gRNA to a promoter active in Gram-positive cells and a terminator active in Gram-positive cells.

In certain embodiments, the DNA break induced by the endonuclease is repaired/replaced with an incoming sequence. For example, to precisely repair the DNA break generated by the Cas9 expression cassette and the gRNA expression cassette described above, a nucleotide editing template is provided, such that the DNA repair machinery of the cell can utilize the editing template. For example, about 500 bp 5′ of targeted gene can be fused to about 500 bp 3′ of the targeted gene to generate an editing template, which template is used by the Gram-positive host's machinery to repair the DNA break generated by the RGEN.

The Cas9 expression cassette, the gRNA expression cassette and the editing template can be co-delivered to filamentous fungal cells using many different methods (e.g., protoplast fusion, electroporation, natural competence, or induced competence). The transformed cells are screened by PCR amplifying the target gene locus, by amplifying the locus with a forward and reverse primer. These primers can amplify the wild-type locus or the modified locus that has been edited by the RGEN. These fragments are then sequenced using a sequencing primer to identify edited colonies.

In yet other embodiments, a modified cell is constructed by random or specific mutagenesis using methods well known in the art, including, but not limited to, chemical mutagenesis and transposition. Modification of the gene may be performed by subjecting the parental cell to mutagenesis and screening for mutant cells in which expression of the gene has been reduced or eliminated. The mutagenesis, which may be specific or random, may be performed, for example, by use of a suitable physical or chemical mutagenizing agent, use of a suitable oligonucleotide, or subjecting the DNA sequence to PCR generated mutagenesis. Furthermore, the mutagenesis may be performed by use of any combination of these mutagenizing methods.

Examples of a physical or chemical mutagenizing agent suitable for the present purpose include ultraviolet (UV) irradiation, hydroxylamine, N-methyl-N′-nitro-N-nitrosoguanidine (MNNG), N-methyl-N′-nitrosoguanidine (NTG), O-methyl hydroxylamine, nitrous acid, ethyl methane sulphonate (EMS), sodium bisulphite, formic acid, and nucleotide analogues. When such agents are used, the mutagenesis is typically performed by incubating the parental cell to be mutagenized in the presence of the mutagenizing agent of choice under suitable conditions, and selecting for mutant cells exhibiting reduced or no expression of the gene.

PCT Publication No. WO2003/083125 discloses methods for modifying Gram-positive (Bacillus) cells, such as the creation of Bacillus deletion strains and DNA constructs using PCR fusion to bypass E. coli. PCT Publication No. WO2002/14490 discloses methods for modifying Bacillus cells including (1) the construction and transformation of an integrative plasmid (pComK), (2) random mutagenesis of coding sequences, signal sequences and pro-peptide sequences, (3) homologous recombination, (4) increasing transformation efficiency by adding non-homologous flanks to the transformation DNA, (5) optimizing double cross-over integrations, (6) site directed mutagenesis and (7) marker-less deletion.

Those of skill in the art are well aware of suitable methods for introducing polynucleotide sequences into bacterial cells (e.g., Gram-negative cells, Gram-positive cells). Indeed, such methods as transformation including protoplast transformation and congression, transduction, and protoplast fusion are known and suited for use in the present disclosure. Methods of transformation are particularly preferred to introduce a DNA construct of the present disclosure into a host cell.

In addition to commonly used methods, in some embodiments, host cells are directly transformed (i.e., an intermediate cell is not used to amplify, or otherwise process, the DNA construct prior to introduction into the host cell). Introduction of the DNA construct into the host cell includes those physical and chemical methods known in the art to introduce DNA into a host cell, without insertion into a plasmid or vector. Such methods include, but are not limited to, calcium chloride precipitation, electroporation, naked DNA, liposomes and the like. In additional embodiments, DNA constructs are co-transformed with a plasmid without being inserted into the plasmid. In further embodiments, a selective marker is deleted or substantially excised from the modified Bacillus strain by methods known in the art. In some embodiments, resolution of the vector from a host chromosome leaves the flanking regions in the chromosome, while removing the indigenous chromosomal region.

Promoters and promoter sequence regions for use in the expression of genes, coding sequences (CDS), open reading frames (ORFs) and/or variant sequences thereof in Gram-positive cells are generally known on one of skill in the art. Promoter sequences of the disclosure are generally chosen so that they are functional in the Gram-positive cells. For example, promoters useful for driving gene expression in Bacillus cells include, but are not limited to, the B. subtilis alkaline protease (aprE) promoter, the α-amylase promoter (amyE) of B. subtilis, the α-amylase promoter (amyL) of B. licheniformis, the α-amylase promoter of B. amyloliquefaciens, the neutral protease (nprE) promoter from B. subtilis, a mutant aprE promoter, or any other promoter from B licheniformis or other related Bacilli. Methods for screening and creating promoter libraries with a range of activities (promoter strength) in Bacillus cells is describe in Publication No. WO2002/14490.

### IV. FERMENTING GRAM-POSITIVE CELLS FOR THE PRODUCTION OF PROTEINS

As generally described above, certain embodiments are related to compositions and methods for constructing and obtaining Gram-positive cells having increased protein production phenotypes. Thus, certain embodiments are related to methods of producing proteins of interest in Gram-positive cells by fermenting the cells in a suitable medium. Fermentation methods well known in the art can be applied to ferment Gram-positive cells of the disclosure.

In some embodiments, the cells are cultured under batch or continuous fermentation conditions. A classical batch fermentation is a closed system, where the composition of the medium is set at the beginning of the fermentation and is not altered during the fermentation. At the beginning of the fermentation, the medium is inoculated with the desired organism(s). In this method, fermentation is permitted to occur without the addition of any components to the system. Typically, a batch fermentation qualifies as a “batch” with respect to the addition of the carbon source, and attempts are often made to control factors such as pH and oxygen concentration. The metabolite and biomass compositions of the batch system change constantly up to the time the fermentation is stopped. Within typical batch cultures, cells can progress through a static lag phase to a high growth log phase, and finally to a stationary phase, where growth rate is diminished or halted. If untreated, cells in the stationary phase eventually die. In general, cells in log phase are responsible for the bulk of production of product.

A suitable variation on the standard batch system is the “fed-batch” fermentation system. In this variation of a typical batch system, the substrate is added in increments as the fermentation progresses. Fed-batch systems are useful when catabolite repression likely inhibits the metabolism of the cells and where it is desirable to have limited amounts of substrate in the medium. Measurement of the actual substrate concentration in fed-batch systems is difficult and is therefore estimated on the basis of the changes of measurable factors, such as pH, dissolved oxygen and the partial pressure of waste gases, such as CO₂. Batch and fed-batch fermentations are common and known in the art.

Continuous fermentation is an open system where a defined fermentation medium is added continuously to a bioreactor, and an equal amount of conditioned medium is removed simultaneously for processing. Continuous fermentation generally maintains the cultures at a constant high density, where cells are primarily in log phase growth. Continuous fermentation allows for the modulation of one or more factors that affect cell growth and/or product concentration. For example, in one embodiment, a limiting nutrient, such as the carbon source or nitrogen source, is maintained at a fixed rate and all other parameters are allowed to moderate. In other systems, a number of factors affecting growth can be altered continuously while the cell concentration, measured by media turbidity, is kept constant. Continuous systems strive to maintain steady state growth conditions. Thus, cell loss due to medium being drawn off should be balanced against the cell growth rate in the fermentation. Methods of modulating nutrients and growth factors for continuous fermentation processes, as well as techniques for maximizing the rate of product formation, are well known in the art of industrial microbiology.

In certain embodiments, a protein of interest expressed/produced by a Gram-positive cell of the disclosure may be recovered from the culture medium by conventional procedures including separating the host cells from the medium by centrifugation or filtration, or if necessary, disrupting the cells and removing the supernatant from the cellular fraction and debris. Typically, after clarification, the proteinaceous components of the supernatant or filtrate are precipitated by means of a salt, e.g., ammonium sulfate. The precipitated proteins are then solubilized and may be purified by a variety of chromatographic procedures, e.g., ion exchange chromatography, gel filtration.

In some embodiments, the cells are cultured under batch or continuous fermentation conditions. A classical batch fermentation is a closed system, where the composition of the medium is set at the beginning of the fermentation and is not altered during the fermentation. At the beginning of the fermentation, the medium is inoculated with the desired organism(s). In this method, fermentation is permitted to occur without the addition of any components to the system. Typically, a batch fermentation qualifies as a “batch” with respect to the addition of the carbon source, and attempts are often made to control factors such as pH and oxygen concentration. The metabolite and biomass compositions of the batch system change constantly up to the time the fermentation is stopped. Within typical batch cultures, cells can progress through a static lag phase to a high growth log phase, and finally to a stationary phase, where growth rate is diminished or halted. If untreated, cells in the stationary phase eventually die. In general, cells in log phase are responsible for the bulk of production of product.

A suitable variation on the standard batch system is the “fed-batch” fermentation system. In this variation of a typical batch system, the substrate is added in increments as the fermentation progresses.

Fed-batch systems are useful when catabolite repression likely inhibits the metabolism of the cells and where it is desirable to have limited amounts of substrate in the medium. Measurement of the actual substrate concentration in fed-batch systems is difficult and is therefore estimated on the basis of the changes of measurable factors, such as pH, dissolved oxygen, and the partial pressure of waste gases, such as CO₂. Batch and fed-batch fermentations are common and known in the art.

Continuous fermentation is an open system where a defined fermentation medium is added continuously to a bioreactor, and an equal amount of conditioned medium is removed simultaneously for processing. Continuous fermentation generally maintains the cultures at a constant high density, where cells are primarily in log phase growth. Continuous fermentation allows for the modulation of one or more factors that affect cell growth and/or product concentration. For example, in one embodiment, a limiting nutrient, such as the carbon source or nitrogen source, is maintained at a fixed rate and all other parameters are allowed to moderate. In other systems, a number of factors affecting growth can be altered continuously while the cell concentration, measured by media turbidity, is kept constant. Continuous systems strive to maintain steady state growth conditions. Thus, cell loss due to medium being drawn off should be balanced against the cell growth rate in the fermentation. Methods of modulating nutrients and growth factors for continuous fermentation processes, as well as techniques for maximizing the rate of product formation, are well known in the art of industrial microbiology.

In certain embodiments, a protein of interest expressed/produced by a Gram-positive cell of the disclosure may be recovered from the culture medium by conventional procedures including separating the host cells from the medium by centrifugation or filtration, or if necessary, disrupting the cells and removing the supernatant from the cellular fraction and debris. Typically, after clarification, the proteinaceous components of the supernatant or filtrate arc precipitated by means of a salt, e.g., ammonium sulfate. The precipitated proteins are then solubilized and may be purified by a variety of chromatographic procedures, e.g., ion exchange chromatography, gel filtration.

### V. PROTEINS OF INTEREST

A protein of interest (POI) of the instant disclosure can be any endogenous or heterologous protein, and it may be a variant of such a POI. The protein can contain one or more disulfide bridges or is a protein whose functional form is a monomer or a multimer, i.e., the protein has a quaternary structure and is composed of a plurality of identical (homologous) or non-identical (heterologous) subunits, wherein the POI or a variant POI thereof is preferably one with properties of interest.

For example, in certain embodiments, a modified Gram-positive cell of the disclosure produces at least about 0.1% more, at least about 0.5% more, at least about 1% more, at least about 5% more, at least about 6% more, at least about 7% more, at least about 8% more, at least about 9% more, or at least about 10% or more of a POI, relative to its unmodified (reference or control) cell.

In certain embodiments, a modified Gram-positive cell of the disclosure exhibits an increased specific productivity (Qp) of a POI relative the control cell. For example, the detection of specific productivity (Qp) is a suitable method for evaluating protein production. The specific productivity (Qp) can be determined using the following equation:

$$“ Qp = gP / gDCW \cdot hr ”$$

wherein, “gP” is grams of protein produced in the tank; “gDCW” is grams of dry cell weight (DCW) in the tank and “hr” is fermentation time in hours from the time of inoculation, which includes the time of production as well as growth time.

Thus, in certain other embodiments, a modified Gram-positive cell of the disclosure comprises a specific productivity (Qp) increase of at least about 0.1%, at least about 1%, at least about 5%, at least about 6%, at least about 7%, at least about 8%, at least about 9%, or at least about 10% or more, relative to the unmodified (parental) cell.

In certain embodiments, a POI or a variant POI thereof is selected from the group consisting of acetyl esterases, aminopeptidases, amylases, arabinases, arabinofuranosidases, carbonic anhydrases, carboxypeptidases, catalases, cellulases, chitinases, chymosins, cutinases, deoxyribonucleases, epimerases, esterases, α-galactosidases, β-galactosidases, α-glucanases, glucan lysases, endo-β-glucanases, glucoamylases, glucose oxidases, α-glucosidases, β-glucosidases, glucuronidases, glycosyl hydrolases, hemicellulases, hexose oxidases, hydrolases, invertases, isomerases, laccases, ligases, lipases, lyases, mannosidases, oxidases, oxidoreductases, pectate lyases, pectin acetyl esterases, pectin depolymerases, pectin methyl esterases, pectinolytic enzymes, perhydrolases, polyol oxidases, peroxidases, phenoloxidases, phytases, polygalacturonases, proteases, peptidases, rhamno-galacturonases, ribonucleases, transferases, transport proteins, transglutaminases, xylanases, hexose oxidases, and combinations thereof.

Thus, in certain embodiments, a POI or a variant POI thereof is an enzyme selected from Enzyme Commission (EC) Number EC 1, EC 2, EC 3, EC 4, EC 5, or EC 6.

There are various assays known to those of ordinary skill in the art for detecting and measuring activity of intracellularly and extracellularly expressed proteins.

### VI. EXEMPLARY EMBODIMENTS

Non-limiting embodiments of compositions and methods disclosed herein are as follows:

1. A variant pro-region sequence comprising amino acid substitutions at position 30 and one or more positions selected from 1, 2, 3, 4, 6, 14, 16, 19, 20, 23, 36, 37, 38, 39, 42, 43, 44, 49, 50, 64, 65, 67, 68, 71, 79, 83 and 84, wherein the amino acid positions of the variant pro-region are numbered according to SEQ ID NO: 15.

2. The variant pro-region of embodiment 1, derived from a parent or reference polypeptide with at least 70%, 75%, 80%, 85%, 86%, 87%, 88%, 89%, 90%, 91%, 92%, 93%, 94%, 95%, 96%, 97%, 98%, 99%, or 100% amino acid sequence identity to positions 1-84 of SEQ ID NO: 15.

3. A variant pro-region sequence comprising amino acid insertions of glycine (G) at position 2 and lysine (K) at position 3, and amino acid substitutions at one or more positions selected from 1, 32, 38, 46, 66, 67, 70 and 73, wherein the amino acid positions of the variant pro-region are numbered according to SEQ ID NO: 14.

4. The variant pro-region of embodiment 3, derived from a parent or reference polypeptide with at least 70%, 75%, 80%, 85%, 86%, 87%, 88%, 89%, 90%, 91%, 92%, 93%, 94%, 95%, 96%, 97%, 98%, 99%, or 100% amino acid sequence identity to positions 1-86 of SEQ ID NO: 14.

5. A variant pro-region sequence comprising amino acid insertions of glycine (G) at position 2, lysine (K) at position 3, and alanine (A) at position 4, wherein the amino acid positions of the variant pro-region are numbered according to SEQ ID NO: 30.

6. The variant pro-region of embodiment 5, derived from a parent or reference polypeptide with at least 70%, 75%, 80%, 85%, 86%, 87%, 88%, 89%, 90%, 91%, 92%, 93%, 94%, 95%, 96%, 97%, 98%, 99%, or 100% amino acid sequence identity to positions 1-87 of SEQ ID NO: 30.

7. A variant pro-region sequence comprising amino acid insertions of glycine (G) at position 2, lysine (K) at position 3, and serine (S) at position 4, wherein the amino acid positions of the variant pro-region are numbered according to SEQ ID NO: 29.

8. The variant pro-region of embodiment 7, derived from a parent or reference polypeptide with at least 70%, 75%, 80%, 85%, 86%, 87%, 88%, 89%, 90%, 91%, 92%, 93%, 94%, 95%, 96%, 97%, 98%, 99%, or 100% amino acid sequence identity to positions 1-87 of SEQ ID NO: 29.

9. A variant pro-region sequence comprising amino acid insertions of glycine (G) at position 2, lysine (K) at position 3, alanine (A) at position 4, and alanine (A) at position 5, wherein the amino acid positions of the variant pro-region are numbered according to SEQ ID NO: 31.

10. The variant pro-region of embodiment 9, derived from a parent or reference polypeptide with at least 70%, 75%, 80%, 85%, 86%, 87%, 88%, 89%, 90%, 91%, 92%, 93%, 94%, 95%, 96%, 97%, 98%, 99%, or 100% amino acid sequence identity to positions 1-88 of SEQ ID NO: 31.

11. A variant pro-region sequence comprising a glutamic acid (E) to glycine (G) substitution at position 30 (E30G), a leucine (L) to lysine (K) substitution at position 68 (L68K) and an isoleucine (I) to valine (V) substitution at position 73 (I72V), wherein the amino acid positions of the variant pro-region are numbered according to SEQ ID NO: 32.

12. The variant pro-region of embodiment 11, derived from a parent or reference polypeptide with at least 70%, 75%, 80%, 85%, 86%, 87%, 88%, 89%, 90%, 91%, 92%, 93%, 94%, 95%, 96%, 97%, 98%, 99%, or 100% amino acid sequence identity to positions 1-84 of SEQ ID NO: 15 or SEQ ID NO: 32.

13. A variant pro-region sequence comprising a glutamic acid (E) to glycine (G) substitution at position 30 (E30G), a leucine (L) to lysine (K) substitution at position 68 (L68K) and glutamic acid (E) to isoleucine (I) substitution at position 80 (E80I), wherein the amino acid positions of the variant pro-region are numbered according to SEQ ID NO: 33.

14. The variant pro-region of embodiment 13, derived from a parent or reference polypeptide with at least 70%, 75%, 80%, 85%, 86%, 87%, 88%, 89%, 90%, 91%, 92%, 93%, 94%, 95%, 96%, 97%, 98%, 99%, or 100% amino acid sequence identity to positions 1-84 of SEQ ID NO: 15 or SEQ ID NO: 33.

15. A nucleic acid encoding a variant pro-region of any one of embodiments 1-14.

16. A polynucleotide comprising a variant pro-region nucleic acid of embodiment 15.

17. A polynucleotide comprising an upstream (5′) nucleic acid encoding a variant pro-region of any one of embodiments 1-14 operably linked to a downstream (3′) nucleic acid sequence encoding a protein of interest (POI).

18. A polynucleotide comprising an upstream (5′) nucleic acid encoding a protein signal (secretion) sequence operably linked to a downstream (3′) nucleic acid sequence encoding a variant pro-region of any one of embodiments 1-14 operably linked to a downstream nucleic acid sequence encoding a protein of interest (POI).

19. An expression cassette comprising an upstream (5′) promoter region sequence operably linked to a downstream (3′) polynucleotide of any one of embodiments 16-18.

20. A Gram-positive host cell comprising an introduced polynucleotide of any one of embodiment 16-18 or an introduced cassette of embodiment 19.

21. The variant pro-region of embodiment 1, wherein the one or more amino acid substitutions increase the net charge of the variant pro-region sequence relative to reference pro-region of SEQ ID NO: 15.

22. The variant pro-region of embodiment 3, wherein the one or more amino acid substitutions increase the net charge of the variant pro-region sequence relative to reference pro-region of SEQ ID NO: 14.

23. The variant pro-region of embodiment 21 or embodiment 22, wherein the increase in the net charge is at least 1 (+1) to 4 (+4).

24. The variant pro-region of embodiment 1, comprising an amino acid modification set forth in TABLE 1.

25. The variant pro-region of embodiment 1, wherein the amino acid substitution at position 30 is a glycine (G), a histidine (H) or an asparagine (N).

26. The variant pro-region of embodiment 1, wherein the amino acid substitution at position 1 is a threonine (T).

27. The variant pro-region of embodiment 1, wherein the amino acid substitution at position 2 is an alanine (A) or a proline (P).

28. The variant pro-region of embodiment 1, wherein the amino acid substitution at position 3 is an alanine (A), a cystine (C), a glycine (G), an arginine (R), a serine (S) or a valine (V).

29. The variant pro-region of embodiment 1, wherein the amino acid substitution at position 4 is a glutanine (Q).

30. The variant pro-region of embodiment 1, wherein the amino acid substitution at position 6 is an arginine (R).

31. The variant pro-region of embodiment 1, wherein the amino acid substitution at position 14 is a threonine (T).

32. The variant pro-region of embodiment 1, wherein the amino acid substitution at position 19 is an arginine (R).

33. The variant pro-region of embodiment 1, wherein the amino acid substitution at position 20 is a lysine (K).

34. The variant pro-region of embodiment 1, wherein the amino acid substitution at position 23 is a lysine (K), an asparagine (N) or a glutamine (Q).

35. The variant pro-region of embodiment 1, wherein the amino acid substitution at position 36 is a glycine (G).

36. The variant pro-region of embodiment 1, wherein the amino acid substitution at position 37 is a threonine (T).

37. The variant pro-region of embodiment 1, wherein the amino acid substitution at position 38 is a histidine (H),

38. The variant pro-region of embodiment 1, wherein the amino acid substitution at position 39 is a lysine (K) or a glutamine (Q).

39. The variant pro-region of embodiment 1, wherein the amino acid substitution at position 44 is an isoleucine (I).

40. The variant pro-region of embodiment 1, wherein the amino acid substitution at position 49 is a threonine (T) or a glutamine (Q).

41. The variant pro-region of embodiment 1, wherein the amino acid substitution at position 50 is an arginine (R).

42. The variant pro-region of embodiment 1, wherein the amino acid substitution at position 64 is a lysine (K).

43. The variant pro-region of embodiment 1, wherein the amino acid substitution at position 65 is a lysine (K).

44. The variant pro-region of embodiment 1, wherein the amino acid substitution at position 67 is a serine (S).

45. The variant pro-region of embodiment 1, wherein the amino acid substitution at position 68 is a lysine (K) or a methionine (M).

46. The variant pro-region of embodiment 1, wherein the amino acid substitution at position 79 is a glutamine (Q).

47. The variant pro-region of embodiment 1, wherein the amino acid substitution at position 83 is an isoleucine (I).

48. The variant pro-region of embodiment 1, wherein the amino acid substitution at position 84 is a leucine (L).

49. The variant pro-region of embodiment 3, comprising an amino acid modification set forth in TABLE 2, TABLE 3, TABLE 4, SEQ ID NO: 14, SEQ ID NO: 29, SEQ ID NO: 30, SEQ ID NO: 31, and combinations thereof.

50. The variant pro-region of embodiment 3, wherein the amino acid substitution at position 1 is a threonine (T).

51. The variant pro-region of embodiment 3, wherein the amino acid substitution at position 32 is a glycine (G).

52. The variant pro-region of embodiment 3, wherein the amino acid substitution at position 38 is a glycine (G).

53. The variant pro-region of embodiment 3, wherein the amino acid substitution at position 46 is an isoleucine (I).

54. The variant pro-region of embodiment 3, wherein the amino acid substitution at position 66 is a lysine (K).

55. The variant pro-region of embodiment 3, wherein the amino acid substitution at position 67 is a lysine (K).

56. The variant pro-region of embodiment 3, wherein the amino acid substitution at position 70 is a lysine (K).

57. The variant pro-region of embodiment 3, wherein the amino acid substitution at position 73 is an arginine (R).

58. A variant pro-region comprising an amino acid modification set for in any one of TABLES 1-5, FIG. 1, FIG. 2, FIG. 3, SEQ ID NO: 9, SEQ ID NO: 11, SEQ ID NO: 14, SEQ ID NO: 29, SEQ ID NO: 30, SEQ ID NO: 32, SEQ ID NO: 33, and combinations thereof.

59. The variant pro-region of embodiment 58, derived from a parent or reference polypeptide with at least 70%, 75%, 80%, 85%, 86%, 87%, 88%, 89%, 90%, 91%, 92%, 93%, 94%, 95%, 96%, 97%, 98%, 99%, or 100% amino acid sequence identity to positions 1-84 of SEQ ID NO: 15.

60. A method for producing a heterologous protein of interest (POI) in a Gram-positive bacterial cell comprising (a) introducing into a Gram-positive cell an expression cassette comprising an upstream (5′) promoter operably linked to a downstream (3′) nucleic acid encoding a variant pro-region sequence comprising amino acid substitutions at position 30 and one or more positions selected from 1, 2, 3, 4, 6, 14, 16, 19, 20, 23, 36, 37, 38, 39, 42, 43, 44, 49, 50, 64, 65, 67, 68, 71, 79, 83 and 84, operably linked to a downstream (3′) nucleic acid sequence encoding the POI, wherein the amino acid positions of the variant pro-region are numbered according to SEQ ID NO: 15, and (b) growing/cultivating/fermenting the modified cell under suitable conditions for the production of the POI.

61. A method for producing a heterologous protein of interest (POI) in a Gram-positive bacterial cell comprising (a) introducing into a Gram-positive cell an expression cassette comprising an upstream (5′) promoter sequence operably linked to a downstream (3′) nucleic acid encoding a variant pro-region sequence comprising amino acid insertions of glycine (G) at position 2, lysine (K) at position 3, and amino acid substitutions at one or more positions selected from 1, 32, 38, 46, 66, 67, 70 and 73, operably linked to a downstream (3′) nucleic acid sequence encoding the POI, wherein the amino acid positions of the variant pro-region are numbered according to SEQ ID NO: 14, and (b) growing/cultivating/fermenting the modified cell under suitable conditions for the production of the POI.

62. A method for producing a heterologous protein of interest (POI) in a Gram-positive bacterial cell comprising (a) introducing into a Gram-positive cell an expression cassette of embodiment 19, and (b) growing/cultivating/fermenting the modified cell under suitable conditions for the production of the POI.

63. The method of any one of embodiments 60-62, wherein the cassette further comprises a nucleic acid encoding a pre-protein signal (secretion) sequence operably linked and positioned between the promoter and variant pro-region sequences.

64. The method of any one of embodiments 60-62, wherein the modified cell produces an increased amount of the POI relative to a control Gram-positive cell having an introduced expression cassette comprising the same upstream (5′) promoter sequence operably linked to a downstream (3′) nucleic acid encoding a pro-region sequence of SEQ ID NO: 15 operably linked to a downstream nucleic acid sequence encoding the same POI.

65. The method of embodiment 64, wherein the increased amount of the POI is at least about 0.1% more, at least about 0.5% more, at least about 1% more, at least about 5% more, at least about 6% more, at least about 7% more, at least about 8% more, at least about 9% more, or at least about 10% or more of a POI relative to the control cell.

66. The method of any one of embodiments 60-63, wherein the performance index (PI) value of the modified cell is at least about 1.1, as compared to a control Gram-positive cell having an introduced expression cassette comprising the same upstream promoter sequence operably linked to a downstream nucleic acid encoding a pro-region sequence of SEQ ID NO: 15 operably linked to a downstream nucleic acid sequence encoding the same POI as the modified cell, wherein the modified and control cells are cultivated under the same conditions.

67. The method of any one of embodiments 60-63, wherein the introduced cassette is integrated into the genome of the cell.

68. The method of any one of embodiments 60-63, comprising at least two introduced cassettes.

69. The method of any one of embodiments 60-63, wherein the POT is selected from the group consisting of enzymes, antibodies, receptors, and regulatory proteins.

70. The method of any one of embodiments 60-63, wherein the POI is selected from the group consisting of acetyl esterases, aminopeptidases, amylases, arabinases, arabinofuranosidases, carbonic anhydrases, carboxypeptidases, catalases, cellulases, chitinases, chymosins, cutinases, deoxyribonucleases, epimerases, esterases, α-galactosidases, β-galactosidases, α-glucanases, glucan lysases, endo-β-glucanases, glucoamylases, glucose oxidases, α-glucosidases, β-glucosidases, glucuronidases, glycosyl hydrolases, hemicellulases, hexose oxidases, hydrolases, invertases, isomerases, laccases, ligases, lipases, lyases, mannosidases, oxidases, oxidoreductases, pectatc lyases, pectin acetyl esterases, pectin depolymerases, pectin methyl esterases, pectinolytic enzymes, perhydrolases, polyol oxidases, peroxidases, phenoloxidases, phytases, polygalacturonases, proteases, peptidases, rhamno-galacturonases, ribonucleases, transferases, transport proteins, transglutaminases, xylanases, hexose oxidases, and combinations thereof.

71. The method of claim 70, wherein the protease is a native or variant subtilisin.

72. The method of any one of claims 60-71, wherein the Gram-positive bacterial cell is a Bacillus sp. cell, optionally wherein the Bacillus sp. cell is selected from the group consisting of B. subtilis, B. licheniformis, B. lentus, B. brevis, B. stearothermophilus, B. alkalophilus, B. amyloliquefaciens, B. clausii, B. halodurans, B. megaterium, B. coagulans, B. circulans, B. lautus, B. thuringiensis and Geobacillus stearothermophilus.

### EXAMPLES

Certain aspects of the present disclosure may be further understood in light of the following examples, which should not be construed as limiting. Modifications to materials and methods will be apparent to those skilled in the art. Standard recombinant DNA and molecular cloning techniques used herein are well known in the art (Ausubel et al., 1987; Sambrook et al., 1989).

### Example 1

### Modification of the GG36 Pro Sequences by Site Evaluation Libraries

In the instant example, a variant subtilisin protein (BG46_variant 1; SEQ ID NO: 2) was used as a reporter to monitor protein expression as described herein. More specifically, a DNA fragment comprising an upstream (5′) aprE gene flanking region was operably linked to a polynucleotide construct (e.g., expression cassette) comprising a variant of B. subtilis rrn1-P2 promoter/5′-aprE UTR region DNA sequence, resulting in SEQ ID NO: 1, operably linked to DNA sequence (SEQ ID NO: 4) encoding an AprE signal sequence operably linked to a DNA sequence (SEQ ID NO: 5) encoding a variant pro-region sequence A (SEQ ID NO: 9) operably linked to a DNA sequence encoding the mature BG46_variant 1 (SEQ ID NO: 2) operably linked to a B. amyloliquefaciens BPN′ terminator DNA sequence (SEQ ID NO: 6), which polynucleotide construct was operably linked to a downstream (3′) aprE gene flanking region sequence which includes a kanamycin (kan) gene expression cassette (SEQ ID NO: 7). More particularly, this DNA fragment was assembled using standard molecular biology techniques and was used as template to develop linear DNA expression cassettes comprising one or more pro-region modifications (mutations) described herein.

### Site Evaluation Libraries (SEL) of Pro-Region Sequence A

The amino acid sequence encoding the variant pro-region sequence A (30H, SEQ ID NO: 9) was used as template to create site evaluation libraries (SEL) and has been developed as 4.4 kb fragments by Twist Bioscience HQ (South San Francisco). The linear DNA of the expression cassettes were used to transform competent B. subtilis cells, wherein the transformation mixtures were plated onto LA plates containing 1.8 ppm kanamycin and incubated overnight at 37° C. Single colonies were picked and grown in Luria broth at 37° C. under antibiotic selection.

Sequence analysis was performed to determine unique pro-region sequence A variants that were cherry picked into 96-well microtiter plates (MTPs). For example, the first (1^{st}) amino acid position of the variant pro-region sequence A (SEQ ID NO: 9) is an alanine (“Ala” or “A”), which can be altered (substituted) to all other nineteen (19) naturally occurring amino acid residues, and the second (2^{nd}) amino acid position of the variant pro-region sequence A (SEQ ID NO: 9) is a glutamic acid (“Glu” or “E”), which can be altered (substituted) to all other nineteen (19) naturally occurring amino acid residues, and so on, until all eighty-four (84) positions of pro-region sequence A (SEQ ID NO:9) have been evaluated (e.g., see FIG. 1).

For reporter protein expression experiments, transformed cells were grown in 96-well MTPs in cultivation medium (enriched semi-defined media based on MOPs buffer for 3 days at 32° C., 300 rpm, with 80% humidity in shaking incubator, which were centrifuged and filtrated. Clarified culture supernatants were used to measure (assay) reporter protease activity to determine productivity levels, wherein samples were taken after seventy-two (72) hours. The reporter protease activity assay is further described below in Example 2.

### Positions That Affect Productivity

As set forth below, TABLE 1 shows the results of the relative reporter productivity compared to the reference construct in which the reporter gene was expressed with the native GG36 pro-region sequence (SEQ ID NO: 15), wherein expression was measured by the activity assay described below in Example 2. For example, as shown in TABLE 1, performance index (PI) values are given for samples taken after seventy-two (72) hours (PI calculated as described in Example 2), wherein amino acid (residue) positions that affected the productivity in combination with position 30 are 1, 2, 3, 4, 6, 14, 19, 20, 23, 36, 37, 38, 39, 42, 43, 44, 49, 50, 64, 65, 67, 68, 71, 83, 84. The 5′ amino acid changes, as well as 3′ amino acid modifications impact productivity, in addition 80% of the selected combinations had an extra positive charge.

TABLE 1
PERFORMANCE INDEX PRO-REGION VARIANT
A SEQUENCES COMPARED TO WT PRO

| Variants vs WT | PI | Δ Charge |
|---|---|---|
| None | 1.0 | 0 |
| E30H | 1.5 | 1 |
| E30G | 2.3 | 1 |
| E6R-E30H | 2.5 | 3 |
| S19R-E30H | 2.3 | 2 |
| E30H-E39Q | 2.4 | 2 |
| E23Q-E30H | 2.3 | 2 |
| E3G-E30H | 2.7 | 2 |
| E30H-L44I | 2.2 | 1 |
| E30H-T83I | 2.0 | 1 |
| E30H-E38H | 2.5 | 2 |
| E23N-E30H | 2.1 | 2 |
| E3A-E30N | 2.5 | 2 |
| E30H-E67S | 2.1 | 2 |
| E30H-E39K | 2.7 | 3 |
| E30H-A79Q | 2.2 | 1 |
| E3V-E30H | 2.5 | 2 |
| E3C-E30H | 2.4 | 2 |
| A1T-E30H | 2.5 | 1 |
| A4Q-E30H | 2.6 | 1 |
| E30H-T50R | 2.3 | 2 |
| E30H-L68K | 1.5 | 2 |
| E30H-E37T | 2.4 | 2 |
| E30H-E36G | 2.1 | 2 |
| E23K-E30H | 2.2 | 3 |
| E14T-E30H | 2.1 | 2 |
| E2P-E30H | 2.4 | 2 |
| E2A-E30H | 2.1 | 2 |
| E3R-E30H | 2.7 | 3 |
| E30H-E49T | 2.3 | 2 |
| E3S-E30H | 2.2 | 2 |
| E30H-E49Q | 2.3 | 2 |
| E30H-M84L | 2.2 | 2 |
| E30H-A65K | 2.3 | 2 |
| E20K-E30H | 2.2 | 3 |
| E30H-D64K | 2.3 | 3 |
| E30H-L68M | 1.5 | 1 |
| E30H-E43D | 2.2 | 1 |
| E16S-E30H | 2.4 | 2 |
| E30H-I42V | 2.4 | 1 |
| E30H-A71R | 2.5 | 2 |
| A4N-E30H | 2.2 | 1 |
| E30H-D64A | 2.0 | 2 |
| E3P-E30H | 2.4 | 2 |
| E6Q-E30H | 2.4 | 2 |

### Example 2

### Protease Activity Assay

The protease activity of the BG46_variant 1 subtilisin was determined by measuring the hydrolysis of the synthetic suc-AAPF-pNA peptide substrate. For the AAPF assay, the reagent solutions used were 100 mM Tris pH 8.6, 10 mM CalCl₂, 0.005% Tween®-80 (Tris/Ca buffer) and 160 mM suc-AAPF-pNA in DMSO (suc-AAPF-pNA stock solution; Sigma: S-7388). To prepare a working solution, one (1) mL suc-AAPF-pNA stock solution was added to 100 mL Tris/Ca buffer and mixed. An enzyme sample was added to a microtiter plate (MTP) containing one (1) mg/mL suc-AAPF-pNA working solution and assayed for activity at 405 nm over three-five (3-5) minutes using a SpectraMax plate reader in kinetic mode at room temperature. The protease activity was expressed as mOD/minute. The activity of each variant constructed was measured and compared to the reference construct that was grown in the same plate. By dividing the value of reference sample by the value of variant sample, the performance index (PI).

### Example 3

### Modification of the 5′ GG36 Pro Sequences

In the instant example, a BG46_variant 2 subtilisin (SEQ ID NO: 8) was used as a reporter protein to monitor expression as described herein. More specifically, a DNA fragment comprising an upstream (5′) aprE gene flanking region was operably linked to an polynucleotide construct comprising a variant of (5′) B. subtilis rrn1-P2 promoter/5′-UTR aprE region DNA sequence, resulting in SEQ ID NO: 1 operably linked to a DNA sequence (SEQ ID NO: 4) encoding an AprE signal peptide sequence operably linked to a DNA sequence (SEQ ID NO: 10) encoding variant pro-region sequence B (SEQ ID NO: 9, 30H) operably linked to a DNA sequence encoding the mature BG46_variant 2 subtilisin (SEQ ID NO: 8) operably linked to a B. amyloliquefaciens BPN′ terminator DNA sequence (SEQ ID NO: 6), which polynucleotide construct was operably linked to a downstream (3′) aprE gene flanking region sequence which includes a kanamycin (kan) gene expression cassette (SEQ ID NO: 7). More particularly, this DNA fragment was assembled using standard molecular biology techniques and was used as template to develop linear DNA expression cassettes comprising one or more promoter region modifications described herein.

### Introducing 5′ Pro-Region Modification

The 5′ (N-terminal) sequence of the seventy-eight (78) amino acid AprE pro-region sequence (SEQ ID NO: 12) was used to exchange the first three (3) amino acid residues with the first amino acid residue (A) of the variant pro-region sequence C (SEQ ID NO: 14) resulting in SEQ ID NO: 13. As indicated above in TABLE 1, a mutant of the pro-region variant A sequence (i.e., TABLE 1, “E030G”; SEQ ID NO: 11) showed high expression of the reporter. Standard molecular biology techniques were used to prepare the expression construct and compare to the reference pro-region. The variants (mutants) were developed as 4.4 kb linear DNA fragment and used to transform competent B. subtilis cells, wherein the transformation mixtures were plated onto LA plates containing 1.8 ppm kanamycin and incubated overnight at 37° C. Single colonies were picked and grown in Luria broth at 37° C. under antibiotic selection.

For reporter protein expression experiments, transformed cells were grown in 96-well MTPs in cultivation medium (enriched semi-defined media based on MOPs buffer for 3 days at 32° C., 300 rpm, with 80% humidity in shaking incubator, which were centrifuged and filtrated. Clarified culture supernatants were used to measure (assay) reporter protease activity to determine productivity levels, wherein samples were taken after 72 hours. The reporter protease activity assay was performed as described above in Example 2, wherein protein productivity of the variant pro-region C was about 1.1× higher than the E030G-GG36-Pro (SEQ ID NO: 11).

### Example 4

### Further Modification of the GG36 Pro Sequence

In the instant example, the BG46_variant 2 subtilisin (SEQ ID NO: 8) was used as a reporter protein to monitor expression as described herein. The construction was done according to the description set forth above in Example 3. More specifically, combinatorial libraries were prepared on the reference pro-region sequence (SEQ ID NO: 15) in which the amino acids glycine (G) and lysine (K) were inserted (“GK”) at the 5′ (N-term) sequence (e.g., see FIG. 2, SEQ ID NO: 14).

### Combinatorial Library in Variant Pro-Region C

A selection was made from the TABLE 1 data to combine pro-region mutations, wherein standard molecular biology techniques were used to prepare the expression construct and compare that to the reference pro-region. The linear DNA expression cassettes were used to transform competent B. subtilis cells, wherein the transformation mixtures were plated onto LA plates containing 1.8 ppm kanamycin and incubated overnight at 37° C. Single colonies were picked and grown in Luria broth at 37° C. under antibiotic selection. For reporter protein expression experiments, transformed cells were grown in 96-well MTPs in cultivation medium (enriched semi-defined media based on MOPs buffer for 3 days at 32° C., 300 rpm, with 80% humidity in shaking incubator, which were centrifuged and filtrated. Clarified culture supernatants were used to measure (assay) reporter protease activity to determine productivity levels, wherein samples were taken after 72 hours. The reporter protease activity assay was performed as described above in Example 2.

Combinations in the Pro-Region that Improve Productivity

As set presented below, TABLE 2 reveals sequence results of combinations of pro-region mutations that showed increased productivity of the expressed reporter (SEQ ID NO: 8), wherein the delta (A) charge varies between +1 and +4. More specifically, positive charges as combinations in the loop region (i.e., residue positions 66-73). Performance index (PI) values are given of samples taken after 72 hours, calculated as described above in Example 2. For example, PI values of the pro-region variants are compared to the WT pro-region sequence (TABLE 2, SEQ ID NO: 15), wherein the amino acid position numbering (TABLE 2, 1^{st} column) is in comparison with SEQ ID NO: 14, without “GK” insertion. As shown in FIG. 3, variant pro-region sequences may be numbered according to the position numbering of the reference variant pro-region sequence C (SEQ ID NO: 14; comprising 86 amino acid positions) or the wild-type (reference) pro-region sequence (SEQ ID NO: 15; comprising 84 amino acid positions).

TABLE 2
PERFORMANCE INDEX PRO-REGION VARIANT C (SEQ
ID NO: 14) SEQUENCES COMPARED TO WT PRO
(SEQ ID NO: 15) WITHOUT GK INSERTION

| Variants vs WT | PI | Δ Charge |
|---|---|---|
| NONE | 1.0 | 0 |
| E32G | 1.7 | 1 |
| L46I-D66K-A73R | 1.9 | 3 |
| A67K-L70K-A73R | 1.9 | 3 |
| L70K-A73R | 2.0 | 2 |
| E38G-A67K-L70K | 2.0 | 3 |
| D66K-A67K-L70K | 2.0 | 4 |
| E32G-L70K | 2.0 | 2 |
| A1T-L46I-A73R | 2.0 | 1 |
| L70K-A73R | 2.0 | 2 |
| E38G-L46I-D66K-A67K | 2.0 | 4 |
| E38G-D66K-A67K | 2.0 | 4 |
| L46I-D66K-A67K-L70K | 2.0 | 4 |
| E38G-L46I-A67K | 2.0 | 2 |
| L46I-L70K | 2.1 | 1 |
| L46I-L70K-A73R | 2.1 | 2 |
| E38G-L46I-A67K | 2.1 | 2 |
| L46I-D66K-A67K-L70K | 2.2 | 4 |
| L46I-A67K-L70K | 2.3 | 2 |

### Example 5

Modification of the 5′ GG36 Pro Sequence in Bacillus licheniformis

In the instant example, the BG46_variant 2 subtilisin (SEQ ID NO: 8) was used as a reporter protein to monitor expression as described herein. The construction was generally performed as follows. A first DNA fragment comprising a (5′) serA gene flanking region (5′ serA gene FR) which includes a Bacillus licheniformis serA selectable marker expression cassette (SEQ ID NO: 16) was operably linked to a polynucleotide construct comprising an upstream (5′) B. subtilis rrn1-p3 promoter region DNA sequence (SEQ ID NO: 17) operably linked to a DNA sequence of the B. subtilis aprE 5′ untranslated region (5′-UTR; SEQ ID NO: 18) operably linked to DNA encoding the B. subtilis AprE signal sequence (SEQ ID NO: 4) operably linked to DNA encoding a variant pro-region C sequence (SEQ ID NO: 14) operably linked to a DNA sequence encoding the mature BG46_variant 2 subtilisin (SEQ ID NO: 8) operably linked to a B. licheniformis amyL terminator (SEQ ID NO: 19) operably linked to a (3′) serA gene flanking region (3′ serA gene FR) (SEQ ID NO: 20).

A second DNA fragment comprising a (5′) lysA gene flanking region (5′ lysA gene FR) which includes a B. licheniformis lysA selectable marker expression cassette (SEQ ID NO: 21) was operably linked to a polynucleotide construct comprising an upstream (5′) B. subtilis rrn1-p3 promoter region DNA sequence (SEQ ID NO: 17) operably linked to a DNA sequence of the B. subtilis aprE 5′-UTR (SEQ ID NO: 18) operably linked to DNA encoding the B. subtilis AprE signal sequence (SEQ ID NO: 4) operably linked to DNA encoding a variant pro-region C sequence (SEQ ID NO: 14) operably linked to a DNA sequence encoding the mature BG46_variant 2 subtilisin (SEQ ID NO: 8) operably linked to a B. licheniformis amyL terminator (SEQ ID NO: 10) operably linked to a (3′) lysA gene flanking region (3′ lysA gene FR; SEQ ID NO: 22).

More particularly, these DNA fragments were assembled using standard molecular biology techniques and were used as a template to develop linear DNA expression cassettes comprising one or more pro-region sequence modifications described herein. For example, B. licheniformis strains comprising variant pro-region sequences were constructed by integrating the first and second DNA fragments (described above) in the genome, where the first and second fragments contain the pro-region sequence variants set forth below in TABLE 3. As shown in TABLE 3, production of the reporter protein was determined as described previously using standard methods (e.g., see WO2019/055261) and normalized to the control pro-region sequence containing the E30G variant.

TABLE 3
PERFORMANCE INDEX PRO-REGION VARIANT C (GK, SEQ:
14) SEQUENCES IN B. LICHENIFORMIS COMPARED TO
CONTROL PRO-REGION SEQUENCE B (E32G; SEQ: 11)

| Variant vs WT | PI | Δ Charge |
|---|---|---|
| E32G | 1.0 | 1 |
| E32G-L70K | 1.1 | 2 |
| E32G-A73R | 1.3 | 2 |
| E32G-L70K-A73R | 1.2 | 3 |

### Example 6

### Additional Modifications of the 5′ GG36 Pro Sequence

In the instant example, the BG46_variant 2 subtilisin (SEQ ID NO: 8) was used as a reporter protein to monitor expression as described herein. In particular, the 5′ (N-terminal) sequence of the mutated GG36 PRO sequence (SEQ ID NO:14; Variant Pro-Region Sequence C) consisting of 86 amino acids was further modified to introduce additional amino acids such as alanine (A) or serine (S) after the lysine in position three (3) (e.g., to potentially facilitate the release of the mature protease into the medium). For instance, these N-terminal modifications resulted in the three (3) variant Pro-Region sequences D-F (SEQ ID NO: 29-31) as presented in FIG. 1-3.

These N-terminal modified GG36 PRO sequences (SEQ ID NO: 29, SEQ ID NO: 30 and SEQ ID NO: 31) were assembled in an expression cassette comprising an upstream (5′) aprE gene flanking region operably linked to an polynucleotide construct comprising a variant of (5′) B. subtilis rrn1-P2 promoter/5′-UTR aprE region DNA sequence, resulting in SEQ ID NO: 1 operably linked to a DNA sequence (SEQ ID NO: 4) encoding an AprE signal peptide sequence operably linked to a DNA sequence encoding the variant pro-region sequence operably linked to a DNA sequence encoding the mature BG46_variant 2 subtilisin (SEQ ID NO: 8) operably linked to a B. amyloliquefaciens BPN′ terminator DNA sequence (SEQ ID NO: 6), which polynucleotide construct was operably linked to a downstream (3′) aprE gene flanking region (3′ aprE gene FR) sequence which includes a downstream kanamycin (kan) gene expression cassette (SEQ ID NO: 7). The cassette comprising the mutated/variant PRO sequence was transformed into Bacillus subtilis cells using standard molecular biology techniques.

Transformed cells were grown in 96-well MTPs in cultivation medium (enriched semi-defined media based on MOPs buffer for 3 days at 32° C., 300 rpm, with 80% humidity in shaking incubator, which were centrifuged and filtrated. Clarified culture supernatants were used to measure (assay) reporter protease activity to determine productivity levels, wherein samples were taken after seventy-two (72) hours. In particular, TABLE 4 shows the results of the reporter protein productivity (performance index (P1) values) after 72 hours as compared to the reference construct in which the reporter protein was expressed with the WT PRO sequence (SEQ ID NO: 14).

TABLE 4
PERFORMANCE INDEX PRO-REGION VARIANTS D-F
(SEQ ID NO: 29-31) COMPARED TO VARIANT
C PRO-REGION SEQUENCE (SEQ ID NO: 14)

| Name | Variant | PI | SEQ ID |
|---|---|---|---|
| Variant C | GK | 1.00 | 14 |
| Variant E | GKA | 1.3 | 30 |
| Variant D | GKS | 1.2 | 29 |
| Variant F | GKAA | 1.4 | 31 |

As shown above in TABLE 4, the PI values of the pro-region variants were compared to the pro-region sequence variant pro-region Sequence C (SEQ ID NO: 14, “G₂K₃”), wherein the amino acid position numbering is in comparison with SEQ ID NO: 14 (i.e., with “GK” insertion). The PI index of the three (3) N-terminal pro-region mutants (AGKA, AGKS, AGKAA) showed high expression of the reporter protein BG46_variant 2 subtilisin (SEQ ID NO: 8) after 72 hours of growth compared to variant C (AGK; SEQ ID NO: 14).

### Example 7

### C-Terminal Modifications of GG36 Pro Sequence

In the instant example the GG36 variant pro-region sequence B (SEQ ID NO: 11) was further engineered to substitute the leucine (L) at position sixty-eight (68) with lysine (K) and to substitute the isoleucine (I) at position seventy-two (72) with valine (V), or to substitute the glutamate (E) at position eighty (80) with an isoleucine (I), resulting in the two (2) engineered pro-region sequence variant sequences G (SEQ ID NO: 32) and H (SEQ ID NO: 33), e.g., see FIG. 1-FIG. 3. In particular, transformed cells were grown as described above, and the clarified culture supernatants were used to measure (assay) reporter protease activity to determine productivity levels, wherein samples were taken after 72 hours.

For example, TABLE 5 below shows the results of the reporter protein productivity (PI values) after 72 hours as compared to the reference construct in which the reporter protein was expressed with the variant B pro-region sequence (SEQ ID NO: 11). More particularly, as presented in TABLE 5, the PI values of the variant pro-region sequences G and H were increased as compared to the reference/control variant B pro-region sequence, wherein the variant pro-region sequence G (comprising L68K and I72V mutations) resulted in a PI of 1.08 relative to the pro-region sequence B (E30G), and the variant pro-region sequence H (comprising L68K and E80I mutations) resulted in a PI of 1.18 relative to the pro-region sequence B (E30G).

TABLE 5
PERFORMANCE INDEX PRO-REGION VARIANTS G
AND H COMPARED TO PRO-REGION VARIANT C

| Name | Variant | PI | Δ Charge vs WT |
|---|---|---|---|
| Variant B | E30G | 1.0 | 1 |
| Variant G | E30G L68K I72V | 1.4 | 2 |
| Variant F | E30G L68K E80I | 1.5 | 3 |

### REFERENCES

PCT Publication No. WO1993/20214
PCT Publication No. WO2002/14490
PCT Publication No. WO2003/083125
PCT Publication No. WO2008/112258
PCT Publication No. WO2010/123754
PCT Publication No. WO2016/205710
Altschul et al., “Basic local alignment search tool”, J. Mol. Biol., 215(13):403-410, 1990.
Altschul et al., “Gapped BLAST and PSI BLAST a new generation of protein database search programs”, Nucleic Acids Res, Set 1; 25(17):3389-402, 1997.
Karlin et al., “Applications and statistics for multiple high-scoring segments in molecular sequences”, PNAS USA, 90(12):5873-5787, 1993.
Saitou and Nei, “The neighbor-joining method: a new method for reconstructing phylogenetic trees”, Mol. Biol. Evol., Vol. 4, issue 4, pages 406-425, 1987.
Schaffer et al., “Improving the accuracy of PSI-BLAST protein database searches with composition-based statistics and other refinements”, Nucleic Acids Res, 29(14):2994-3005, 2001.
Thompson et al., “CLUSTAL W: improving the sensitivity of progressive multiple sequence alignment through sequence weighting, position-specific gap penalties and weight matrix choice”, Nucleic Acids Res, 22(22):4673-4680, 1994.
Beaucage and Caruthers, “Deoxynucleoside phosphoramidites—A new class of key intermediates for deoxypolynucleotide synthesis”, Tetrahedron Lett., 22,1859-1862, 1981.
Matthes et al., “Simultaneous rapid chemical synthesis of over one hundred oligonucleotides on a microscale”, EMBO J., 3:801-805, 1984.

1. A variant pro-region sequence comprising amino acid substitutions at position 30 and one or more positions selected from 1, 2, 3, 4, 6, 14, 16, 19, 20, 23, 36, 37, 38, 39, 42, 43, 44, 49, 50, 64, 65, 67, 68, 71, 79, 83 and 84, wherein the amino acid positions of the variant pro-region are numbered according to SEQ ID NO: 15.

2. The variant pro-region of claim 1, derived from a parent or reference polypeptide with at least about 70%, about 75%, about 80%, about 85%, 86%, 87%, 88%, 89%, 90%, 91%, 92%, 93%, 94%, 95%, 96%, 97%, 98%, 99%, or 100% amino acid sequence identity to SEQ ID NO: 15.

3. A variant pro-region sequence comprising amino acid insertions of glycine (G) at position 2 and lysine (K) at position 3, and amino acid substitutions at one or more positions selected from 1, 32, 38, 46, 66, 67, 70 and 73, wherein the amino acid positions of the variant pro-region are numbered according to SEQ ID NO: 14.

4. The variant pro-region of claim 3, derived from a parent or reference polypeptide with at least 70%, 75%, 80%, 85%, 86%, 87%, 88%, 89%, 90%, 91%, 92%, 93%, 94%, 95%, 96%, 97%, 98%, 99%, or 100% amino acid sequence identity to SEQ ID NO: 14.

5. A variant pro-region sequence comprising a glutamic acid (E) to glycine (G) substitution at position 30 (E30G), a leucine (L) to lysine (K) substitution at position 68 (L68K) and an isoleucine (I) to valine (V) substitution at position 73 (I72V), wherein the amino acid positions of the variant pro-region are numbered according to SEQ ID NO: 15, or a variant pro-region sequence comprising a glutamic acid (E) to glycine (G) substitution at position 30 (E30G), a leucine (L) to lysine (K) substitution at position 68 (L68K) and glutamic acid (E) to isoleucine (I) substitution at position 80 (E80I), wherein the amino acid positions of the variant pro-region are numbered according to SEQ ID NO: 15.

6. The variant pro-region of claim 5, derived from a parent or reference polypeptide with at least 70%, 75%, 80%, 85%, 86%, 87%, 88%, 89%, 90%, 91%, 92%, 93%, 94%, 95%, 96%, 97%, 98%, 99%, or 100% amino acid sequence identity to SEQ ID NO: 15.

7. A polynucleotide comprising the variant pro-region nucleic acid of claim 5.

8. A polynucleotide comprising an upstream nucleic acid encoding a variant pro-region of claim 5 operably linked to a downstream nucleic acid sequence encoding a protein of interest (POI).

9. A polynucleotide comprising an upstream nucleic acid encoding a signal sequence operably linked to a downstream nucleic acid sequence encoding a variant pro-region of claim 5 operably linked to a downstream nucleic acid sequence encoding a protein of interest (POI).

10. An expression cassette comprising an upstream promoter operably linked to a downstream polynucleotide of claim 9.

11. A Gram-positive host cell comprising an introduced cassette of claim 10.

12. A method for producing a protein of interest (POI) in a Gram-positive bacterial cell comprising:
introducing into a Gram-positive cell an expression cassette comprising an upstream promoter operably linked to a downstream nucleic acid encoding a variant pro-region sequence comprising amino acid substitutions at position 30 and one or more positions selected from 1, 2, 3, 4, 6, 14, 16, 19, 20, 23, 36, 37, 38, 39, 42, 43, 44, 49, 50, 64, 65, 67, 68, 71, 79, 83 and 84, operably linked to a downstream nucleic acid encoding the POI, wherein the amino acid positions of the variant pro-region are numbered according to SEQ ID NO: 15, and
cultivating the modified cell under conditions for the production of the POI.

13. A method for producing a protein of interest (POI) in a Gram-positive bacterial cell comprising:
introducing into a Gram-positive cell an expression cassette comprising an upstream promoter sequence operably linked to a downstream nucleic acid encoding a variant pro-region sequence comprising amino acid insertions of glycine (G) at position 2, lysine (K) at position 3, and amino acid substitutions at one or more positions selected from 1, 32, 38, 46, 66, 67, 70 and 73, operably linked to a downstream nucleic acid encoding the POI, wherein the amino acid positions of the variant pro-region are numbered according to SEQ ID NO: 14, and
cultivating the modified cell under conditions for the production of the POI.

14. (canceled)

15. The method of claim 12, wherein the modified cell produces an increased amount of the POI relative to a control Gram-positive cell having an introduced expression cassette comprising the same upstream promoter sequence operably linked to a downstream nucleic acid encoding a pro-region sequence of SEQ ID NO: 15 operably linked to a downstream nucleic acid sequence encoding the same POI.

16. The method of claim 12, wherein the increased amount of the POI is at least about 0.1% increased relative to the control cell.

17. The method of claim 12, wherein the POI is selected from the group consisting of acetyl esterases, aminopeptidases, amylases, arabinases, arabinofuranosidases, carbonic anhydrases, carboxypeptidases, catalases, cellulases, chitinases, chymosins, cutinases, deoxyribonucleases, epimerases, esterases, α-galactosidases, β-galactosidases, α-glucanases, glucan lysases, endo-β-glucanases, glucoamylases, glucose oxidases, α-glucosidases, β-glucosidases, glucuronidases, glycosyl hydrolases, hemicellulases, hexose oxidases, hydrolases, invertases, isomerases, laccases, ligases, lipases, lyases, mannosidases, oxidases, oxidoreductases, pectate lyases, pectin acetyl esterases, pectin depolymerases, pectin methyl esterases, pectinolytic enzymes, perhydrolases, polyol oxidases, peroxidases, phenoloxidases, phytases, polygalacturonases, proteases, peptidases, rhamno-galacturonases, ribonucleases, transferases, transport proteins, transglutaminases, xylanases, hexose oxidases, and combinations thereof.


SEQUENCE LISTING (33 sequences)

SEQ ID NO: 1 - DNA, 980 bp, Bacillus subtilis
  source 1..980: mol_type = other DNA
ctgacgatattgcctcctgcttttccggccagaccatctttgtaattaatgccggaataagcaactttaatcaggacaccatccttcggcaaatcctctgttgatatggttttcacatggactgaaacatcatcggcatttttttctgcctgcaaggcttgaaataacgttgacattcggcacactccttttcatttatatcgtaaccgaagaacgttcaaaaaaccaaatcatcaagccgccattttcacttcgccggcacattgagacaataatggacaaatccggtatcctcttcatagccgttttgctcatacaagcttcttgccttccggttgtggtgctcagtctgaagtgttaaacattttgccccgttttgccctgcataatcctttgcggcagaaagcagccggccgccggctccctttgtacgcgcatgaggaacgacaaataagtcatttaatatgtatatccttttcattgacacagaagaaaacgttggatagagctgggtaaagcctatgaattctccattttcttctgctatcaaaataacagactcgtgattttccaaacgagctttcaaaaaagcctctgccccttgcaaatcggatgcctgtctataaaattcccgatattggttaaacagcggcgcaatggcggccgcatctgatgtctttgcttggcgaatgttcatcttatttcttcctccctctcaataattttttcattctatcccttttctgtaaagtttatttttcagaatacttttatcatcatgctttgaaaaaatatcacgataatatccattgttctcacggaagcacacgcgctgataaacagctgacatcaactaaaagtttcattaaatactttgaaaaaagttgttgacttaaaagaagctaaatgttatagtaattgtacagaatagtcttttaagtaagtctactctgaatttttttaaaaggagagggtaaaga

SEQ ID NO: 2 - protein, 269 aa, Bacillus gibsonii
QQTVPWGITRVQAPAVHNRGITGSGVRVAILDSGISAHSDLNIRGGASFVPGEPTTADLNGHGTHVAGTVAALNNSIGVIGVAPNAELYAVKVLGANGSGSVSGIAQGLEWAATNNMHIANMSLGSDFPSSTLERAVNYATSRDVLVIAATGNNGSGSVGYPARYANAMAVGATDQNNRRANFSQYGTGIDIVAPGVNVQSTYPGNRYVSMNGTSMATPHVAGAAALVKQRYPSWNATQIRNHLKNTATNLGNSSQFGSGLVNAEAATR

SEQ ID NO: 3 - protein, 269 aa, synthetic construct
QQTVPWGITRVQAPAVHNRGITGSGVRVAILDSGISAHEDLNIRGGASFVPGEPTTADLNGHGTHVAGTVAALDNSIGVIGVAPNAELYAVKVLGANGRGSVSGIAQGLEWAATNNMHIANMSLGAEGPSSTLERAVNYATSRDVLVIAATGNNGSGSVGYPARYANAMAVGATDQNNRRANFSQYGTGIDIVAPGVNVQSTYPGNRYVSLNGTSMATPHVAGAAALVKQRYPSWNATQIRNHLKNTATNLGNSSEFGSGLVNAEAATR

SEQ ID NO: 4 - DNA, 87 bp, Bacillus subtilis
  source 1..87: mol_type = other DNA
gtgagaagcaaaaaattgtggatcagcttgttgtttgcgttaacgttaatctttacgatggcgttcagcaacatgtctgcgcaggct

SEQ ID NO: 5 - DNA, 252 bp, synthetic construct
  source 1..252: mol_type = other DNA
gctgaagaagcaaaagaaaaatatttaattggctttaatgagcaggaagctgtcagtgagtttgtagaacaagtagaggcaaatgaccatgtcgccattctctctgaggaagaggaagtcgaaattgaattgcttcatgaatttgaaacgattcctgttttatccgttgagttaagcccagaagatgtggacgcgcttgaactcgatccagcgatttcttatattgaagaggatgcagaagtaacgacaatg

SEQ ID NO: 6 - DNA, 247 bp, Bacillus amyloliquefaciens
  source 1..247: mol_type = other DNA
acataaaaaaccggccttggccccgccggttttttattatttttcttcctccgcatgttcaatccgctccataatcgacggatggctccctctgaaaattttaacgagaaacggcgggttgacccggctcagtcccgtaacggccaagtcctgaaacgtctcaatcgccgcttcccggtttccggtcagctcaatgccgtaacggtcggcggcgttttcctgataccgggagacggcattcgtaatc

SEQ ID NO: 7 - DNA, 2007 bp, synthetic construct
  source 1..2007: mol_type = other DNA
agtctttcgactgagcctttcgttttatttgatgcctcaagctagagagtcattaccagatctcactgcagtcactaaaacaattcatccagtaaaatataatattttattttctcccaatcaggcttgatccccagtaagtcaaaaaatagctcgacatactgttcttccccgatatcctccctgatcgaccggacgcagaaggcaatgtcataccacttgtccgccctgccgcttctcccaagatcaataaagccacttactttgccatctttcacaaagatgttgctgtctcccaggtcgccgtgggaaaagacaagttcctcttcgggcttttccgtctttaaaaaatcatacagctcgcgcggatctttaaatggagtgtcttcttcccagttttcgcaatccacatcggccagatcgttattcagtaagtaatccaattcggctaagcggctgtctaagctattcgtatagggacaatccgatatgtcgatggagtgaaagagcctgatgcactccgcatacagctcgataatcttttcagggctttgttcatcttcatactcttccgagcaaaggacgccatcggcctcactcatgagcagattgctccagccatcatgccgttcaaagtgcaggacctttggaacaggcagctttccttccagccatagcatcatgtccttttcccgttccacatcataggtggtccctttataccggctgtccgtcatttttaaatataggttttcattttctcccaccagcttatataccttagcaggagacattccttccgtatcttttacgcagcggtatttttcgatcagttttttcaattccggtgatattctcattttagccatttattatttccttcctcttttctacagtatttaaagataccccaagaagctaattataacaagacgaactccaattcactgttccttgcattctaaaaccttaaataccagaaaacagctttttcaaagttgttttgaaagttggcgtataacatagtatcgacggagccgataacgcctcactcctcacatcaacccgttacttctattgtaatcataaattcaaattcttagaaccaagctgtgttccgcacttttccacccttttaagcatggaaaccccgatcgctgggaaaactaacaatgtttggagtgatgcaaatgaaaaaaatagtggcagccatcgtggtaatcggtcttgtgtttatcgcatttttttatctttacagccgatcaggcgatgtgtatcaatcggtagacgcggatttgatcacactgtcttcaagcggccaggaagatatcgagattgaaaaaagacagcacgtcaaagatatgctggatattatgaatcagggaaaacaggtgaagacagaaaaaacatcagcccctgattacgaagggacaatcaagtttcataaagaccggtatgactcattcagactatggattgacggcagccagcaagccgtttttttgaaggatggcacatactacaaattaagcaaaaatgatacaaaggcgctgctaaatattattaaaaaagaagcaaaggattgaaaatgaaaaagcgaagctaaccgcttcgctttttcattttattggggcaaaatatctctcagtgcccgtctgagcattttccccgtcgcatttttcggaatatcgtcaagaaacgtaatggcggcaggccgcttgtattttgccagatgcttttcgcagtgctgcatgatgtcctcctctgttaccccagagcgtttcggcaccacatatccctttaccgcttccccgctttgggggtccggcacgccgatgacaaccgcctccttgacgtccggatggctgtacagcacctcctccacctcccgcggatacacattgtatcctcctacaatgatcatgtcttttttccggtcaacaatgtaaaaatagccgtcctcatcccgtcttgccaagtcccccgtataaagccacccgtctttta

SEQ ID NO: 8 - protein, 269 aa, synthetic construct
QQTVPWGITRVQAPAVHNRGITGSGVRVAILDSGISAHEDLNIRGGASFVPGEPTTADLNGHGTHVAGTVAALDNSIGVIGVAPNAELYAVKVLGANGRGSVSGIAQGLEWAATNNMHIANLSLGAEGPSSTLERAVNYATSRDVLVIAATGNNGSGSVGYPARYANAMAVGATDQNNRRANFSQYGTGIDIVAPGVAVQSTYPGNRYVSQQGTSMATPHVAGAAALVKQRYPSWNATQIRDHLKNTATNLGNSSQFGSGLVNAEAATR

SEQ ID NO: 9 - protein, 84 aa, synthetic construct
AEEAKEKYLIGFNEQEAVSEFVEQVEANDHVAILSEEEEVEIELLHEFETIPVLSVELSPEDVDALELDPAISYIEEDAEVTTM

SEQ ID NO: 10 - DNA, 252 bp, synthetic construct
  source 1..252: mol_type = other DNA
gctgaagaagcaaaagaaaaatatttaattggctttaatgagcaggaagctgtcagtgagtttgtagaacaagtagaggcaaatgacggcgtcgccattctctctgaggaagaggaagtcgaaattgaattgcttcatgaatttgaaacgattcctgttttatccgttgagttaagcccagaagatgtggacgcgcttgaactcgatccagcgatttcttatattgaagaggatgcagaagtaacgacaatg

SEQ ID NO: 11 - protein, 84 aa, synthetic construct
AEEAKEKYLIGFNEQEAVSEFVEQVEANDGVAILSEEEEVEIELLHEFETIPVLSVELSPEDVDALELDPAISYIEEDAEVTTM

SEQ ID NO: 12 - protein, 77 aa, Bacillus amyloliquefaciens
AGKSNGEKKYIVGFKQTMSTMSAAKKKDVISEKGGKVQKQFKYVDAASATLNEKAVKELKKDPSVAYVEEDHVAHAY

SEQ ID NO: 13 - DNA, 258 bp, synthetic construct
  source 1..258: mol_type = other DNA
gccggaaaagaagaagcaaaagaaaaatatttaattggctttaatgagcaggaagctgtcagtgagtttgtagaacaagtagaggcaaatgacgaggtcgccattctctctgaggaagaggaagtcgaaattgaattgcttcatgaatttgaaacgattcctgttttatccgttgagttaagcccagaagatgtggacgcgcttgaactcgatccagcgatttcttatattgaagaggatgcagaagtaacgacaatg

SEQ ID NO: 14 - protein, 86 aa, synthetic construct
AGKEEAKEKYLIGFNEQEAVSEFVEQVEANDEVAILSEEEEVEIELLHEFETIPVLSVELSPEDVDALELDPAISYIEEDAEVTTM

SEQ ID NO: 15 - protein, 84 aa, Bacillus lentus
AEEAKEKYLIGFNEQEAVSEFVEQVEANDEVAILSEEEEVEIELLHEFETIPVLSVELSPEDVDALELDPAISYIEEDAEVTTM

SEQ ID NO: 16 - DNA, 4569 bp, Bacillus licheniformis
  source 1..4569: mol_type = other DNA
ctgggagctgatagcacagcgatttcccgcctcctgtcggaagcatcgccaccgtgtcccgtccctccagcacgcttttgatgatcgtttcctggccttttttaaaagcatgataaccaaaatacctgcggagcgcctgatgaagtttatccattcctgatcaccgttttcgacagggccaggcggattttaaaataactgtatcggccgccgagtccctctttaatttgtctgattttattggtttggttcgctatcgcataatcggcaataattttctgttcctctttcgacacgtactgatcgatggaaaaagacggatcatgaatggcgatttccacaatgtgatcctcgatcgtcgccaatttcagctttctaatttttgcgatccggtctattgtatacccttcttttataagaaagagcgtttttctcgtcgattgtgtcagaccatcattcaaaggaatgtcatgaatgagcgtttgaaaaagcggactttcaccattttgggcggattgaataaaatggtgcagaacatcccaaaacagtgcatatatgtaccactcatcaaggttcattttttcggaaagctgtctgaatgtatagcccgcttttgttctggatgtcagggagtgaacaaaaatagcggcctgttcatcatgatttaaaacagacagcttctctttcagctcttggtgaaactgggccgctgtttcagcagcattccggtttcttaaatactgtttaacccagttctgaatctgataatcttttacgatcggcaaatacacgcgttcgcggtaaagcttgttggagagcacttggatcaaaagcgacatccttgcccacatcactttggctgccgcctgataatagccgccgtgaaaatggcggggccacggataaagggcaaaaaagccggcaagttccgcttctcctttttcggtgacggtgtaagcgccgctttccgctttttctctgaccaaagactcctgcttcaatcgttgaacgctggctgccacctgctctctcgacagagccgaacaaaagccgaagtattttgaaacggcaaataaaccggcgtcctgtatcgtctgtgacgacctttttccttttaataaatgatagaccgcgcttggagaacgctcacccttcatggatgacagaatgtcaagcacaatcgcgtcaaaaaaatgaaccggcatatcatcacctgcaatcttccggcaacattcgatcatttcttccttttattttaacagattttgcggagaaatcgacgtttaaactcatataaaaggggtatgttagcagtagaacccttgtgtgataagcattctcaatatttttgagttgaaatgtaagattaacaccattacaataaggaatgggaataggtttcatatcggatagatagagggttaaaccatttgttccaacgaagaacaatctgggaggttttttattcatgccaaaatatacaattgtagacaaagatacgtgcatcgcatgcggagcttgtggtgctgcggctcctgatatttatgattacgacgatgagggaatcgcatttgtcacccttgacgacaatcagggtgtcgtcgaagtccctgacgtcttagaagaagacatgatggacgcgtttgaaggctgtcctacagattcgatcaaagttgcggatgagccgttcgaaggcgacccgcttaaacacgaataaagccaaaaaacatccggtgcacaaagtgccggatgtttttttatgagataagcacggctttaccaacaagcaaaaagaagccggctaaagacatccggcttcttctgcagctgacaatatccgggaacatgcacccgatattgtcatgtttatttatttggccatgcggacgttttccttcagccgcggtttcagcgaaaggaaaatcggcgtggacacgagggccacagcgatgcctttaatgaaattaaaaggcaggattccggccagaactgttgtcttgagcgcctctccagtcagcgctggagcatttaaaaaccaagtgtaggcaggcagaaacagcagataatttaaaatgctcatcgaaacggccatcacaagcgtccctgcgaaaagagctgtgacaaaccctttggcagaacttgattttttcagcagtacagctgccggcaggataaacaatgttccggcaatgaagttagccgcctgatcaatcggaacgcccgaggcgcttcctgcaataaagtaattcagcacgtttttgatcgcttcaacggcaatcccggctcccggaccgtacaaaataacagcgagcaatgccgggatatcactgaaatcgatttttaaatacgggaatgcccccaggatcggaaagctcagcatcattaaaataaatgcgatgctgctcagcatgctgatagagacgagacgtctcaccttgttgtgtttcattttgtcactctctccttttcgatcacatctcacgaaaagaggaatggttctttcccctgtcctaaacaaaaaacccgctttattgaaaaagcggggctgttttacagacaggtcaaataaacgtttgaaaatgttcatttcaaaacgcgcggaacctccatcttctcccatccagactatactgtcggcttcggaatcgcaccgaatcctgcccataaaaaggctcgcgggcttagagcgcttgctcatcaccgccggtagggaatttcaccctgccccgaagattgatcttatttatttttaatactgatattattataaattaattgtgaaaaaatgtacaggtgcaaagcttattgcgctgttttgggacatcctgcacgatatttcggtaaactcactttttccgcatactaaaaaccgcacattcacagttatttcatttttaattttcgtctttccgcgtgaaactcattgacactctttatggaatatggtaaattatcagatatttatgacgcttatttaggaggaaatcttacatgtttcgagtattggtctcagataaaatgtccagcgacggcctcaaaccattaatggaagcagattttattgaaattgtagaaaagaatgttgcggaagcggaagacgagcttcatacgtttgacgcgctcttggtgcggagcgccacgaaggtaaccgaagagctgtttaaaaagatgacttcgctgaaaatcgtcgccagagcaggtgtcggcgtcgacaatatcgatattgacgaggcgacaaaacacggtgttatcgtcgtaaacgcgccaaacgggaatacaatttcaaccgctgaacatacctttgcaatgttttcagcgttaatgagacatattccgcaggcaaacatctccgtgaaatcaagggagtggaatcgttcggcttacgtcggttcagagctttacggaaaaacgctcggcatcatcggaatgggccgcatcggaagcgaaatcgcgagccgcgcaaaagcattcggtatgaccgttcatgtatttgacccgttcctgacccaagaaagggcaagcaagctcggcgttaacgcgaacagctttgaagaagttctggcatgcgccgacatcattacggttcataccccgctcacgaaagaaacgaagggacttttgaacaaagaaaccatcgcaaaaacgaaaaaaggcgttcgtctcgttaactgtgcaagaggcggcatcatcgatgaagcagcgcttttggaagctctggaaagcggacatgtcgctggcgctgccttggatgtattcgaagtcgagcctccggtcgattcaaaactgatcgatcatccgcttgtagtcgcgactcctcacttgggcgcctcaacaaaagaagcccagctgaatgtcgctgcacaagtgtccgaagaagtccttcagtatgcgcaaggaaaccctgtgatgtccgcgatcaaccttccggccatgacaaaggattcattcgaaaaaatccagccttatcatcagtttgccaatacgatcggaaaccttgtgtctcagtgcatgaatgagcctgttcaagatgtagccatccaatatgaaggctccatcgccaaacttgaaacgtcatttattacgaaaagccttttggccggatttctgaagccgagggtcgcggctaccgttaacgaagtgaatgccggcaccgttgcgaaagagcgcggcatcagcttcagcgaaaaaatttcttccaatgagtcaggctatgaaaactgcatctctgtgactgtcacgggagatgtaacaacattctctttaagagcgacgtacattccgcacttcggcggacgcatcgttgccttaaacggctttgatattgatttttatccggctggacaccttgtctacattcaccaccaggataaaccaggggctatcggccatgtcggacgaattttaggagaccatgacatcaatatcgccactatgcaggtaggccgaaaagaaaaaggcggagaagcgatcatgatgctttcctttgaccgccaccttgaggacgatattttagctgagctgaaaaacatcccggatatcgtgtctgttaaagccatcgaccttccttaa

SEQ ID NO: 17 - DNA, 95 bp, synthetic construct
  source 1..95: mol_type = other DNA
gtcgctgataaacagctgacatcaatatcctattttttcaaaaaatattttaaaaagttgttgacttaaaagaagctaaatgttatagtaataaa

SEQ ID NO: 18 - DNA, 58 bp, Bacillus subtilis
  source 1..58: mol_type = other DNA
acagaatagtcttttaagtaagtctactctgaatttttttaaaaggagagggtaaaga

SEQ ID NO: 19 - DNA, 34 bp, Bacillus licheniformis
  source 1..34: mol_type = other DNA
cggatttcctgaaggaaatccgtttttttatttt

SEQ ID NO: 20 - DNA, 3186 bp, Bacillus licheniformis
  source 1..3186: mol_type = other DNA
ggaacctgaaaagaattcctttcaggttccgttttttttaggaattctccctgatctcaagcatctggcggggataaatccgctctcctttcaaatcgttccattctttgaggcgctgtacagttacgcccattttttcggcgatatgatgaagcgtatcccctttccgcactacatatgtaccggtcttcgattcatcgtcatgaaggcggagtgtttggccggccttgagatttgaatgtttcaacccgtttattctcatgatctcctcgatggatataccgctatccttgctgattctccagagcgtgtcccctttttgaacggtcaccgcaccgctcattgtcccggcgttttgataaacgtggatagaattttgccggaacgcctcctcacgaagcaccgtcagcggattgattgcatatcttttatcttcagtccatgaaccgtgatgcatttcaaaatgcaggtgggttccggtcgatattcccgtattgccgatgattccgatttgctcgccttttttcacccgctccttttcctttttcaggcgtttgcttaagtgggcataaacggtttcatatccgttgtcatgtttaataaatatcacttggccgtaggagtcggattgatacgatttgcttatcgttccgtctgcggctgccgctactgcttccccttcgggagcagcgatgtcaagccccttatgctttccgcctctcgtaccgaattgatctgtgatctctcctttaatcggttcaatccactctgaggcttccgcccccggggcattgacgaaaagcgccaatcccgaaagccatgcgatcgcgaacaggaagttttgatgtctgagtttcttcaaggttttccatatcctcctattacatgcatcttcggtaaaattgccccctattcggagacagcttagtatacttccaaatcaatacaatttatacattaaaaaaagactccgcacagggagtcttttagttttctatcgtcatcggattcggtgcgtacggaacctgtacagatttcgacaggtcataggcgccgaccttggttatggatgcgtttttaaatttcacttttgtgaagccgaaatctttcgcggtcaatagaaggccttccaccatcaagacatcttcgggtttattttcaatattcgcggaggaagaaaattgaatgatcagttcttttccattcttttgaatatcttcaatcggcgtatcatcggataaaatgggttttaaatgagtgccgctttcttcgtttttcatcatcttaatcgcttcctgcaccgattcgtaagattcgcttgaaggtgcaaggaaccggcgcccgtctgagctttcatataaatagtagcatttttgcgtctggtgcataatcgccatatcggcgagcattccgaatgtttcaaattcaacacccgatttatcattggaaataaacagaacagaatcatacgatccccatttaaaggtttcgttgatcacatttttcagccgttcgaaatcttcgactgatagctccggtattttctcatcaacttgaatcttcagttttttattgtttttctgctctttgaacttcaccttatcaaggtaagctgtgtcaaatgatgtaaactggtccactccaagccggctgtaagcgtgaagcgcatcttcaagatttgtcatgccagtgcttttctcgaggcttaccgggacaacgacagacttggactcgtcaaggaaagcgaaggtgatatagtcgtctttttgattctgtgagacgacaaacgtatttgcaggttcagacttggcagcatcagcctccgtctgcaccaattttccgtcagaagaaatgttggcgtcggcgctgttttgagatctgatctgttcgattaactggggagtgatcagcatcagaagaaagagaaccaaaactgtagcagcaaatgcgccgacccgttttttcggtgatttacgctttggtgcgagaatcagcttttgatagatctgatttgccgaacgattatccttaaccgttggaagttggcttagtaacgccttcagccgttcttcgttccattctgacttcttcattctttggatcctccttcaaaagctccatctgtttacgaagcactttcagaccgcggtgctgagtggtttttaccttgctttcggaaaaattcaaggcttttgctgtttcactgatcgaatatccttgaataaaacgcaagacgataactgatctttggtcaagcgtacacttgtctagggcctcgaaaatttcctttaggttttcattttgcatcacgatgtcctcaggcagaggcttgcggtcttttacatcttgtttctcccagtcaaacgtccccaaaatccgctggcggatcgtctgctgctttctgaaccagtcgatcgcaacgtgccgcgcaatcgaaagaagccaggttttttcgctgctcctgccttcaaatgtttcgtaagaatgcaggacgcggatgtatacttcctgaactaagtcttccgcctgatttttgtcttttaccatataaaataaaaactgaaataaatcctgatgatactgatcatatattttctgaaaggtttcttccacctgaaacccctccgttcaatttattgtcgtttgtcaatcttaaaaggttacattacaactattacaactatattacgaacatatgaaaatggaaagggggttttgcgaaagttaagcttaattttaacttaacaagcacaaaagcacccgttctaaatgaacaggtgccaaggttataggagcccacattttcactaagctgtgcccttacaaggctttcgttctcctgaccggagcgttgcggatccgctgaaatgaactaatttcaatccgtttatgactttaagtccaattgttggcgaagctttttggaaatctccattctcttttcgtcagtcactaggtgataccataagccgtcaacttttttgtcttcaccttcaatttctacctgcttaatatctttagcagcttctttataagaactttgaatatcgatcatcttgtccagtgttaagtttgttttgatgtttttttctaatgctgccaaaatctcttgatagtttgttaatgattttaattgtgcaccttcatgaataatttcgttgataatctcacgctgacgctcttgac

SEQ ID NO: 21 - DNA, 4450 bp, Bacillus licheniformis
  source 1..4450: mol_type = other DNA
catcggacagctcttgcttgatatcttcaaaatgacgccggctcatgtcatgtcaacttttgtcgtatctggagcgatccttgacggattcggcatttacgaccgttttatcgaatttgccggtgccggggctacagtcccgattgtcagcttcggccactctcttttgcacggcgcgatgcaccaggctgagaaacatggctttatcggaatcggcatggggatatttgaactgacatctgccggtatatctgccgctatcttgttcgcttttcttgttgccgtgatttttaaaccgaaaggataaaggaaaatgccagcaaaacgcaaggtcattttggtcacagacggcgatatatacgctgcaaaagcaatcgaatatgcagcaagaaaaacgggtggccgctgcatttcccaatcggcggggaatccgagcgttaaaacaggaccggagcttgtaaccatgatcctgcaaacccctcatgatcctgtattcgtcatgtttgatgattccggacttcaaggtgaaggcccgggagagacagctatgaaatatgtagcgatgcatcccgatatcgaggtgctcggagtcatcgccgtcgcttcaaaaactcattatgcagagtggacgagagtcgatgtatcaatcgatgcagaaggcgaactgacagagtacggcgtcgataaacacggggtcaaagagttcgatgtcaaacgaatgaatggtgatacagtctattgccttgaccagctggatgttccgatcattgtcggaatcggtgatatcggtaagatgaacagaaaagacgatgtggaaaaaggttcgccgattacaatgaaagcggtcgagctcattttagaaaggagcgggtatcatgagtgctcaaaagcaagagaagacgaacgtattccttgatccttctaagaatgaagcgtatttcaagaagcgggtcggcatgggagaaagctttgaccttggcgtacggaaggtctttattctcggacatgaagttcagctttattatgtcaacggattgtgcgacacacaatacatcattcacctgttaagagaactggtgcatctgaatgataaagaaaaagaatcgggcgaggtcgaagacatcgtcgaaaacaggcttttgaaccagcaggtttcaaaagcggaaacgcttgatgaagctgtcgaccaagtgttgtcaggactggttgccatcatcgtcgaagatgcgggctttgcttttatcatcgatgtcagaagctacccgggcagaacgccggaagaacctgatacagaaaaagtcgtacgcggtgcaagggacggactcgtcgagaacatcatcgtcaacacagccctgattagacgccggatcagagatgagcgcttgcgctacaaaatgcttcatatcggtgaacgctctaaaacagacatctgcctctgctatttggaagacgttgcagatcccgatcttgttgaagtattaaaaaaagaaattgaagatgtgaagatcgacgggctgccgatgtcggataaatcggtagaggaattcctggtcggccaaggctacaatccgtttccgcttgtcaggtttacggaaagggcagacgtagccgcaagccatattttagaggggcatgtcatcgtgatcgtcgatacgtcgccaagcgtcatcatcacaccgaccactttgtttcaccatgttcagcatgctgaggaatacagacagacgccggctgttgggacgtttttaaggtgggtgcggtttttcggtattttggcctccacctttttgctgccgctttggctgctgtttgtcattcatccgtcgctcttgcctgataatttatcgtttatcgggttgaataaagacacccatattccgattatcatgcagattttcctggcggatctcggcgtcgaatttttaagaatggccgccattcatacgccgacggcgctttcgactgcaatgggcctgatcgccgctgtattgatcggcgatatcgcgatcaatgtcggcttgttttctcccgaagtcattttatacgtttccctctcggcaatcggagcctacacgacaccaagctacgagctgagcctggcgaataaaatggtgaagctgtttatgctgatattggtggcgctttttaaagtggagggatttgtcatcggattaacgatcttaactatagtgatgacttcgatcaggtcattgcgaacgccttacttatggcctctcctcccgttcaatggaaaagcgttttggcatgttctcgtgcgcacgtccgttccagggggaaaagtcaggccgagcatcgttcatccgagaaaccgctccagacagccgtgaagccggcattcgaagaggcttttccccggggaaaagcctctttttcaataatcgaattccggtctttgagtaccgatgcctctgtattcattggcagagatcgcgactgcccggaggctgcagatgttgttctgtcttctgatcggatagacgacatacagcatttcgcggccgtacgggtcaatcgttgacgaatgaaggaaaacctcagttcctctccgccaaaatctcgtattcgccggagctgtaataatctgcccttcataaggctcataaattctctgttcataatgcgcagccggctgataaggggcgtatacatcttcaggtgcatagccgggagcgggggtgtagggatagcgatttggatacatatgataacctctttcccacttcgttttttggttttcatctttaagattatattcaggtaaatgcctatttgtatgggcgaaaatctcagcttttcggctctttttttattgaatggacgttgtgtatgcctatttctatcaagcgctgttttctgttattctataatcaatagaatggattagttgtttagggaatcatttcctttataaatcaagaaaatttggacaaatggtggtttagtttttaaaacgaaatgttataatacaacataagaatcgcactatcatgaagccggaagatgcatcgggcagcaaccggagcgccccttgcacctttgtcgatagagaaagagggaatgacaattgtttttacacggtactagcagacaaaatgaaagagggcacctcgaaatcggcggtgtcgatgttctatcattggcagaaagatacggaacacctctttatgtatacgatgtcgcgctgattagagagcgcgcccgaaaattccagaaggcattcaaggaagccggtttaaaagcgcaggtagcgtatgcaagcaaggcgttttcatcggttgccatgattcagcttgccgaacaagaggggctgtctctggatgtggtatcgggaggagagcttttcactgcgatcaaagcagggttcccagctgagcggattcattttcacggaaacaataagagccctgaagaactagccatggcgctggagcatcaaatcggctgcatcgtgctcgataactttcacgagatcgccattacagaagatctttgcaagcgatcaggacaaactgtagacgttttgctcagaatcactccgggagttgaagcgcacacgcacgattatattacgacggggcaggaagattccaaattcggttttgatctgcataatggacaggtcgaacaagccatcgaacaagtcctccgctcgtctgcgtttaagctcctcggcgtgcactgccacatcggttcgcaaatttttgatacggcaggatttgtccttgcagcagacaagattttcgagaagcttgcggaatggcgggagacttactctttcattccggaagtgctcaatcttggcgggggcttcggcatccgctatacaaaagacgacgagccgcttgcagctgatgtttatgttgaaaaaatcatcgaggcggtcaaagcaaatgccgagcatttcggctttgacatccctgagatttggatcgaaccaggccggtctctcgtcggtgatgcggggactacgctgtacacgatcggttctcaaaaagaggtgccgggcattcgcaaatatgtagccatcgacggcggcatgagcgataatatcaggccggcgctttatgaggcaaaatatgaagcagccgtcgccaacaggatgaacgatgcttgtcatgataccgcatcaatcgcaggaaaatgctgcgaaagcggagatatgctgatttgggatttggaaatccccgaagttcgcgacggagatgtgctcgccgttttctgcaccggtgcgtacggctacagcatggccaacaactacaaccgcattccgcgcccggccgtcgtctttgtcgaggacggggaagcgcagctcgtcattcagagagagacgtatgaggatatcgtcaagctggatctgccgctgaaatcgaaagtcaaacaataaaaaaatggagattccctaagaggggggtctccatttttaattcaa

SEQ ID NO: 22 - DNA, 3019 bp, Bacillus licheniformis
  source 1..3019: mol_type = other DNA
caagcacgaaaaacacttcccggtgatcgggaggtgttttttgttaaaaagatcatgacatgcatagaacagcgaccgggctaattgtatataatattgtgaatttaacaaaaaatttacaaaggagatgataaaggcaatgaccagggtgaaaaggatgagatttgctgatttgttggatttagaggcggagtagatgaaaccggccaaagtatccctactccaccgattgctccagtgcctgaagcaatgtgttgattgtaacacagtaaatcgttttacagcaataaacatttttgtgaatattttattgatttcggctgtgatctcattcccatattctgctgcggcccatggcgcaacacagtccggcgatcaatattcaagctttgaagaattggagcggaatgaagatccagcttcttaccgaattacggagaagaacgcaagagtgccgatgctcatcatggccatccatggaggcggcatcgaacccggaacgagcgaaatcgccaatgaagtgtccaaaaactattccctgtacttgtttgaagggctgaaatcatcaggcaatacggaccttcacattacaagcacgcgttttgacgagccagcggcgctcgcaattactgcaagccaccagtatgtcatgtcgctccacggctattacagtgaagaccgcgatattaaagtaggcggcacagaccgcgctaaaatcagaatattggttgatgagctgaaccgctcggggtttgccgctgaaatgctggggacagatgacaagtatgccggaacccatccgaataacatcgccaacaagtcgctttccgggctgagcattcagcttgaaatgagcacgggtttccgcaaatctttattcgaccggtttacactaaaagacagggcggcgacgcaaaacgaaacgttttaccgatttacaaagctgctgacagattttattcatgaaaactatgaagaagacggaggggatttcccctctgcaaaaataaaacacccccttcaagtgaaaaaggaggtgtttcggcggttgtgttaaccgttggactctgaggtgccgccgccggtgaatacggaaacgatggcgttccacagagacacaaagaagtcgatcagtttttgaagaaagttttgtccttcttcagaatccaagaatttcgtgattttatcctttgctttgtcaagctggtctccaacctggttccagtcgatattaatatttttcatgttattaaataaagatataagagagtttttctgatcttctgtgagtgtcacgccaagttcggaagcagccgaatcaatcgttttctccaattcctcttttgactcgggaactccgtttttcgagatttcttccttgactttggccatcagcgctgacgcgttttcactgccgattttctcgccaagctctgaagtggtgacaagctcttcattcgcgacctttttcacatcttcggaaattttttcgcccgaagtcgtttcatacgctttcatcaatccggttaaagcggctgtgcctgacacttcaaacggagcggtgacatagactttggcgtcttttacaccggccgtcatcagcgcgttcaaatacatctcatctgtaattctgctgatattgtgtgtctgaacttccaaaccggtgccttttttcgctacggtaattgaagaagaagaaatcgctcttgttccgatttgtgctttcggtatataatcccctaaatatttatgctcctcatcatttgtcacctcgatgatggtcgcattttcaggcgcattcatttcttttaatactttttgtctgtcctggcttgacaagtctttccccagcgtgacgatgacatcacccactgcggcgtcagcgaagctgacctgcgggaaaatgagcagacacaatgctgtaaagattcctagtatcgattttttcaagctcaatgccctccttaaaaatgcaggcttcaggcagaattgctgtacttttaaagaagcctgccggaacggaaataatgcgttccgaaatatagacggatgaaagatgagtgaggtttcaaagaaaaaaagagagaattttctcttcaagtcaaatgccctcccggcatcgtatctcgccgctcttttatcattcatgattttcacaggcgattcaacctttttttaaaattttttacaaaaacgatacaagagcggcgtttatttcggtcgattggctctctgcttcttcaatatgatataatgacccttgtgaaatgaaaggagagaatcaagatggctaaaaaaggatacatacaactgacaaacggcaaaaaaatcgagtttgaactatatccggatgcggcgccgggaactgtcgccaactttgaaaaacttgcaaacgaagggttctatgacgggctgaagttccaccgcgtcatcccgggcttcgtcagccagggaggctgcccgcacggcaccggaacaggcggacctggatatacgattaaatgcgagacagaagggaatccgcacaaacacgaagccggttctctctcaatggctcacgcaggaaaagataccggaggcagccaattttttatcgtccatgagcctcagccgcacttgaacggcgttcacaccgttttcggaaaggtcacatcaggccttgatgccgtcacttcaatggagcagggacaaggcatggaaaaagtcgaagtatttgatgcataatcagagagcgcaaaaaacagcccgcttagccgggctgtttttttgtctgtaacggtgtttattttccaggtgcaacaggacttgaggccgattcttcgtccacatcctgataggaaataacgatgctaataaataaaataattgtgaaaaaatgaccctttatgtaaaatatattcaagtgaagagctagatagagaacgcaatctgtaaaaaaggaaggggcgtaaggggtgagcgtaaaaatcccatcgacggcagtcggcgtaaaaattaatgactggtataacgcgatacg

SEQ ID NO: 23 - DNA, 261 bp, synthetic construct
  source 1..261: mol_type = other DNA
gccggaaaagcggaagaagcaaaagaaaaatatttaattggctttaatgagcaggaagctgtcagtgagtttgtagaacaagtagaggcaaatgacgaggtcgccattctctctgaggaagaggaagtcgaaattgaattgcttcatgaatttgaaacgattcctgttttatccgttgagttaagcccagaagatgtggacgcgcttgaactcgatccagcgatttcttatattgaagaggatgcagaagtaacgacaatg

SEQ ID NO: 24 - protein, 87 aa, synthetic construct
AGKAEEAKEKYLIGFNEQEAVSEFVEQVEANDEVAILSEEEEVEIELLHEFETIPVLSVELSPEDVDALELDPAISYIEEDAEVTTM

SEQ ID NO: 25 - DNA, 264 bp, synthetic construct
  source 1..264: mol_type = other DNA
gccggaaaagcggctgaagaagcaaaagaaaaatatttaattggctttaatgagcaggaagctgtcagtgagtttgtagaacaagtagaggcaaatgacgaggtcgccattctctctgaggaagaggaagtcgaaattgaattgcttcatgaatttgaaacgattcctgttttatccgttgagttaagcccagaagatgtggacgcgcttgaactcgatccagcgatttcttatattgaagaggatgcagaagtaacgacaatg

SEQ ID NO: 26 - protein, 88 aa, synthetic construct
AGKAAEEAKEKYLIGFNEQEAVSEFVEQVEANDEVAILSEEEEVEIELLHEFETIPVLSVELSPEDVDALELDPAISYIEEDAEVTTM

SEQ ID NO: 27 - DNA, 261 bp, synthetic construct
  source 1..261: mol_type = other DNA
gccggaaaaagcgaagaagcaaaagaaaaatatttaattggctttaatgagcaggaagctgtcagtgagtttgtagaacaagtagaggcaaatgacgaggtcgccattctctctgaggaagaggaagtcgaaattgaattgcttcatgaatttgaaacgattcctgttttatccgttgagttaagcccagaagatgtggacgcgcttgaactcgatccagcgatttcttatattgaagaggatgcagaagtaacgacaatg

SEQ ID NO: 28 - protein, 87 aa, synthetic construct
AGKSEEAKEKYLIGFNEQEAVSEFVEQVEANDEVAILSEEEEVEIELLHEFETIPVLSVELSPEDVDALELDPAISYIEEDAEVTTM

SEQ ID NO: 29 - protein, 87 aa, synthetic construct; identical to SEQ ID NO: 28

SEQ ID NO: 30 - protein, 87 aa, synthetic construct; identical to SEQ ID NO: 24

SEQ ID NO: 31 - protein, 88 aa, synthetic construct; identical to SEQ ID NO: 26

SEQ ID NO: 32 - protein, 84 aa, synthetic construct
AEEAKEKYLIGFNEQEAVSEFVEQVEANDGVAILSEEEEVEIELLHEFETIPVLSVELSPEDVDALEKDPAVSYIEEDAEVTTM

SEQ ID NO: 33 - protein, 84 aa, synthetic construct
AEEAKEKYLIGFNEQEAVSEFVEQVEANDGVAILSEEEEVEIELLHEFETIPVLSVELSPEDVDALEKDPAISYIEEDAIVTTM